(19) United States
(12) Patent Application Publication
(10) Pub. No.: US 2015/0320794 A1
(43) Pub. Date: Nov. 12, 2015

(54) Treatment for candidia infections

(71) Applicant: Karen C. Swenholt, Annandale, VA (US)

(72) Inventor: Karen C. Swenholt, Annandale, VA (US)

(21) Appl. No.: 13/573,545

(22) Filed: Sep. 21, 2012

Related U.S. Application Data
(60) Provisional application No. 61/573,198, filed on Sep. 23, 2011.

(51) Int. Cl. A61K 33/14

(52) U.S. Cl. CPC A61K 33/14

(57) ABSTRACT

A method for treating fungal infections using a metal salt of chlorite alone or in combination with a conventional antifungal agent is disclosed. Treatment is either topical or systemic.

### PRIORITY CLAIM

This application claims priority to U.S. Provisional Patent Application 61/573,198 filed Sep. 23, 2011. The entire content of it is incorporated by reference.

### BACKGROUND OF THE INVENTION

About 7-10% of Americans suffer from onychomycosis, a fungal infection of the nail, particularly the toenail. More than 90 percent of cases are caused by one of two pathogens: Trichophyton rubrum or Trichophyton mentagrophytes, which is also primarily responsible for Tinea Pedis. Factors that have an important effect on the development of onychomycosis include increasing age; genetic susceptibility; and the presence of certain disease states such as diabetes, acquired immunodeficiency syndrome, or peripheral arterial disease. Furthermore, once infected, a secondary infection by other microbes, such as Aspergillus versicolor, sometimes occurs which complicates the condition. Of particular concern are diabetics who are nearly three times more likely to develop onychomycosis than non-diabetics. It has been reported that up to one third of diabetics develop nail fungus. In diabetics, slow healing, particularly in the feet, is common and foot infections of all types can be difficult to treat and may end in amputation.

Feet exposed to a warm, dark, moist environment can get infected. Some people may already be genetically predisposed to onychomycosis. For those who are susceptible, the condition is highly contagious. The best way to avoid onychomycosis is to keep feet clean and dry, washing them at least once a day and drying the toes well. Shoes and socks should be changed daily and should also be kept dry.

Onychomycosis is a medical condition with cosmetic impact because it disfigures the nails. Nails can become thick, discolored, loose, brittle, hard, yellow and painful. Nails are epithelial structures derived from primitive epidermis made up of keratinous fibrils. Once nails are infected, even if an apparently healthy nail is grown out, the infection may remain and the condition relapses. Furthermore, healthy nails can be re-infected.

There appears to be no certain cure for onychomycosis. Current treatments include systemic medications such as itraconazole, terbinafine, ciclopirox and fluconazole, respectively sold under the names Sporonox®, Lamisil® and Diflucan.® Unfortunately, such medications do not eradicate the problem for many patients. Instead, such treatments typically take months to return to a healthy color and are potentially damaging to the liver. Moreover, through the course of treatment patients oftentimes re-infect themselves. Indeed, only about 12% of patients treated with Sporonox have fungus-free nails after one year. Infected nails may also be debrided (cut and thinned) with uncertain results. As a last resort, infected nails may be surgically removed.

Current systemic medications like Lamisil and treatments like the recently marketed light therapy advise patients that they must wait approximately a year before the goal of healthy looking nails is achieved. The remaining diseased nail which grows out at a rate of only 0.75 mm/month is unsightly. In the case of Lamisil, it systemically treats the nail at the site of its growth where the original untreated portion the growing nail contains active fungus. Such a treatment sets the stage for reinfection if treatment stops prematurely. This treatment reduces or eliminates existing fungus under the nail, thus reducing relapse. Quick results encourage compliance with systemic treatments and satisfies the short term expectations of light therapy patients waiting for their nails to grow out.

Light Therapy occasionally requires debridement of the nail surface which necessitates anesthesia. This composition chemically debrides nails without resorting to painful mechanical means, making it a valuable adjunct for developing technologies well into the future.

Urea has the cosmetically undesirable effect of dissolving infected portions of the nail. With treatment according to this invention, infected portions of the nail remain intact and are restored to healthy appearance. Fungus is compromised, not the nail. It is not a safe practice to use Urea directly with an antifungal. Compounds described herein at recommended concentrations carry antifungals to the nail matrix without harming the nail or periungal tissue.

Current topical medications claim to “remove keratin debris” from under nails but are not effective. This invention breaks down the fungal wall at the nail edge, bringing treatment in from the nail edge as well as the top.

Many oralpharyngeal and esophageal fungal infections are caused by Candida albicans. Such infections are typically treated topically with clotrimizole, nystatin, miconazole or butoconazole for initial treatments of oralpharyngeal infection. For later treatments or esophageal infections, systemic treatment is preferred with fluconizole or another azole such as itraconazole or ketoconazole. However, with prolonged treatment, particularly in the immunosuppressed, resistant Candidia albicans and other species of Candida tend to dominate such as Candida glabrata (Torulopsis galbrata), which tends to result in a refractory mucosal candidiasis in 4-5% of immunosuppressed individuals. For fluconizole resistant infections, Amphotericin B (oral or IV), triazoles such as voriconazole or posaconazole, anidulafungin, caspofungin or micafungin may be used. Unfortunately relapses are common and most of these compositions cause or are suspected of causing birth defects and may not be used in pregnant women.

Candidia albicans and other Candida species, C. parapsilosis, C. tropicalis, C. krusei, and C. lusitaniae also cause an invasive systemic infection having serious consequences. Antifungal resistance in Candida can create refractory infections having very serious consequences. It has been estimated that over 10% of infections acquired in an ICU are caused by Candida. Mortality rates have been estimated at between 25 and 75% depending on how it is attributed to the death.

Other fungal infections have similar treatment with the same group of antifungal agents and similar problems with resistance. These include: Cryptococcus neoformins causing a meningitis, Pneumocystis carinii or Pneumocystis jirovecii species causing a pneumonia, Histoplasma capsulatum causing disseminated disease from a pneumonia, and is particularly dangerous if spread to the CNS, Coccidioides immitis causing pneumonia and sometimes disseminated infection, a few different species of Aspergillis cause a pneumonia, Blastomyces dermatitidis, causing pulmonary infection, Sporothrix schenckii, causing skin infections, Tinea species, causing various skin infections, Paracoccidioides brasiliensis, causing a systemic infection, Zygomycetes. Causing a systemic infection and Trichophyton rubrum, causing skin and nail infections.

Many species causing Microsporidiosis, a gastrointestinal infection, are also treated with a wide variety of antifungals and sometimes result in resistance.

Fungal infections tend to reoccur and are difficult to treat in patients with compromised immune systems, diabetes, steroid treatments, chemotherapy, very old and very young.

Other antifungal agents such as griseofulvin, tolfanate and terbinifine have also been used for minor fungal infections.

Many of the same fungi that infect humans also infect animals. Other fungi also infect animals and plants causing great economic harm.

### SUMMARY OF THE INVENTION

It is an object of the present invention to provide a method and composition for treating fungal infections.

It is an also an object of the present invention to provide a method and composition for treating internal and systemic infections as well as topical fungal and bacterial infections of the skin, hair, scalp.

It is another object of the present invention to treating infections generally includes the application of chemical compositions to the nail and surrounding tissues

The present invention method treats infections generally including the application of chemical compositions topically, orally, adsorption across mucus membranes, transdermally or parenterally to the infected and surrounding tissues, or by delivering a composition systemically.

The compositions of the present invention include a salt of chlorite alone or in combination with an antifungal and/or an oil.

It is an also an object of the present invention to provide a method of treating internal and systemic infections of organs and organ systems.

It is yet another object of the present invention to provide a skin healing composition which aids the healing and prevents the infection when the skin is broken.

It is still a further object of the present invention to prevent and treat fungal infections in plants and animals.

The present invention prevents and treats fungal infections by using a composition containing a metal salt of chlorite either alone or in combination with known antifungal compositions.

### DETAILED DESCRIPTION OF THE INVENTION

Unless defined otherwise, all technical and scientific terms used herein have the same meaning as commonly understood by one of ordinary skill in the art to which the invention belongs. Although any methods and materials similar or equivalent to those described herein may be used in the practice or testing of the present invention, the preferred methods and materials are described herein. All references cited herein, including published or corresponding U.S. or foreign patent applications, issued U.S. or foreign patents, and any other references, are each incorporated by reference in their entireties, including all data, tables, figures, and text presented in the cited references.

The term “nail conditions,” as used herein, means conditions present anywhere on or under the nail bed including the cuticle that include, but not limited to, nail flaking, nail splitting, tough thickened nails, nail breakage, weathering damage, brittle nails, yellowing nails, paronychia, nail fungus or onychomycosis, bacterial based infection, and cracked nails.

The term “skin conditions,” as used herein, means a condition present anywhere on the skin including, but not limited to acne, and all manifestations associated with acne including but not limited to comedones, enlarged pores and the like. The term “skin conditions,” also includes but is not limited to blotches; atrophy, dermatitis (including, but not limited to seborrheic dermatitis, erysipelas, erythrasma, eczema, folliculitis, keratoses, melasmas; nodules, nummular dermatitis, precancerous lesions, pruritus, spider veins; senile purpura, warts, wrinkles, sun damaged skin, contact dermatitis, atopic dermatitis, exfoliative dermatitis, perioral dermatitis, psoriasis, and stasis dermatitis), rosacea, impetigo, and other inflammatory skin conditions; minor injury, fissures, and the like.

The term “mucosal conditions” as used herein refers to topical conditions on the mucus membranes, particularly those resulting from fungal infections such as vaginal, oralpharyngeal and esophageal infections.

The term “internal conditions” as used herein means non-topical. Internal infections includes those limited to a single organ such as the lung in pneumonia or infections that spread to multiple organs or even throughout the entire body as in a septicemia.

The present invention relates to methods and compositions for topically treating infections on the surfaces of the body. These compositions may be used in the eye, and on the scalp, skin, hair, wounds and the like for the purposes of improving the cosmetic condition thereof and inhibiting and treating microbial infections. The compositions of the present invention may also be used to treat fungal infections, particularly those of the nails including onychomycosis. The compositions of the present invention may be also be used to treat animal diseases of eye, nail, hoof, hair, and skin.

Sodium chlorite is an antimicrobial agent. It is primarily used for industrial applications such as controlling microbial contamination in industrial cooling systems and towers. It is an oxidizer. Food-processing companies use it for washing fruits and vegetables because it is a fungicide. Since it can destroy natural color matter without attacking the fibers themselves, it is used as a bleaching agent on textiles. When added to an acid, it forms chlorine dioxide, which is used in many municipal water systems to kill microorganisms. Sodium chlorite is currently being used as an ingredient in personal care products such as facial cleansing lotions, acne creams, mouthwash, toothpaste and contact lens preparations for its antimicrobial action and as a preservative in treatments for the eye. Other salts of chlorite have similar uses such as calcium chlorite as a pool shock sanitizer.

In the present invention, alkali and alkaline metal ions are preferred as the salt of chlorite. However, any other cation, whether monovalent, divalent, trivalent, transition metal or other cation may be used such as the ammonium, potassium, magnesium, aluminium, etc. For the purposes of this specification, the chlorite salt is usually referred to by sodium chlorite and the concentrations are weight percentages based on the sodium salt of chlorite. Other chlorite salts may also be substituted for all purposes of the present invention and would have corresponding percentages sufficient to provide the same quantity of chlorite ions.

While measuring sodium chlorite's microbiocidal effectiveness in vitro, creators of previous art determined that the use of chlorite salts in concentrations above 0.75 to 1% would not increase the effectiveness of chlorite salts as an antimycotic. Consequently, only concentrations of less than one percent—sometimes as low as 0.10% have been used as preservatives in compositions and for treatment against fungus infections and other dermatological conditions. Surprisingly, when percentages above 1% were tested in vivo, it was discovered that in addition to its inherent antimycotic effects, sodium chlorite uniquely enhanced penetration of bioactive compounds without irritating skin or nails.

A preferred embodiment of the present invention includes the use of combinations of chemical compositions containing at least one chlorite salt and at least one other antifungal agent. Examples of other antifungal agents include those of the azole class in effective concentrations, including but not limited to itraconazole, butoconazole, clotrimazole, econazole, miconazole, terconazole, tioconazole, luliconazole, lanoconazole, ketaconazole or any similar antimycotic(s), particularly those previously known as antifungal agents or those known to inhibit the biosynthesis of ergosterol and cause changes in oxidative and peroxidative enzyme activities leading to an intracellular buildup of toxic concentrations of hydrogen peroxide.

Another preferred method of the present invention includes the use of hydrogen peroxide and combinations of chemical compositions containing at least one chlorite salt. The hydrogen peroxide may be in any concentration, preferably about 0.75% to 3%, more preferably about 2%. Preferred methods of this invention also include at least one antifungal, preferably of the of the imidazole class.

Another preferred method of the present invention also includes the use of hydrogen peroxide and combinations of chemical compositions containing at least one chlorite salt, in any concentration, preferably above 0.75% , preferably around 2%. This may be used in combination with at least one antifungal such as that of the allylamine class, such as terbinafine.

Depending on the use, formulations for treating the body may have concentrations as low as about 0.001% and as high as about 10%. It should be noted that chlorite ions are many times less toxic than chlorine dioxide to animal cells (Svecevicius et al, Environ Sci Pollut Res Int. 2005 Sep;12(5):302-5, Karrow et al, Drug Chem Toxicol. 2001 Aug;24(3):239-58) and that maintaining it at a higher pH will allow for a higher concentrations to be used. Concentrations of up to 0.5% in internal fluids have been shown to be acceptable for internal use. Higher dosages are likely to be acceptable also. External use allows for considerably higher concentrations to be used, and even higher concentrations are acceptable for short-term contact. The upper limit topically is the tolerance to skin irritation, which will vary with the area of skin being treated and the individual. Concentrations of 100 ppm have been shown to be non-toxic to human cells. Ingram et al, Free Radic Res. 2004 Jul;38(7):739-50. Likewise when used as a disinfectant, higher concentrations are acceptable.

The processes of the present invention may be used in conjunction with one another not only as a method of treating onychomycosis and other dermatological conditions, and also to control symptoms, to prevent onychomycosis and other dermatological conditions and to maintain the health of the nails after diminishing and controlling the symptoms of those conditions.

The processes and compositions of the present invention may be used in conjunction with one as a method of treating or preventing internal conditions, particularly those caused by fungal infections. As serious fungal infections are frequently hospital acquired or result in particularly susceptible people (immunocompromised, undergoing chemotherapy, transplantation, diabetic, those with circulation problems (PAD, phlebitis, . . . ), burns and other tramatic injury, the very young and very old, etc.). These individuals may be treated prophylactically.

The compositions of the present invention may also be used as disinfectants, particularly for medical equipment that is reused or used for extended periods of time. For example, respiratory equipment, facemasks and lines are prone to be contaminated by various fungi. Even if sterilized, these and other medical devices rapidly become contaminated by aerosols from the environment or other patients in a hospital setting. Likewise, sterile and sanitary manufacturing facilities have need for disinfectants, particularly directed towards fungi. These include pharmaceutical and medical diagnostic and device manufacturing, food processing, cosmetic manufacturing, fermentation, microbial and biotechnology production and the packaging facilities for each.

Compositions of the present invention may be used in a number of other items and for uses other than treatment of infection. Prophylactic and sanitary purposes are most common such as toothpaste, mouthwash and other oral hygiene, douches, swabs, soaks, wound dressings, socks, toe socks and the like and portions thereof.

While this specification is directed towards human use, animal or veterinary uses are equally applicable.

In a previous patent application Ser. No. 12/587,495, the contents of which are incorporated by reference, sodium chlorite is taught to be used at concentrations above 1%, preferably around 2% acts as a penetration enhancing agent for the nail and skin. It further teaches that benzoyl peroxide combined with more than 1%; preferably 2% sodium chlorite forms an effective antifungal that is especially powerful against the dermataphytes that cause athlete's foot and onychomycosis. Additionally, these compositions are especially effective when combined with topical antifungal and antibiotic agents.

Conventional antifungal agents are generally fungistatic. However, when chlorite salts are used alone or in conjunction with antifungal agents, the effect is noted to be fungicidal.

In the present invention and possibly hinted in patent application number 20070104798, incorporated herein by reference, sodium chlorite is pharmacologically stable at pH levels that do not create chlorine dioxide. It also notes that combinations of peroxides and sodium chlorite are synergistically effective against microorganisms at pH levels, are pharmacologically stable and that do not create chlorine dioxide.

As shown in the examples, in vitro data was generated and the minimum inhibitory concentrations (MIC) tests were determined in broth culture using ATCC MYA 4438. The results are shown in FIG. 1. Itraconazole is the azole used in the study. On the Y-axis are dilutions of the sodium chlorite where undiluted X contains 6.4 micrograms/ml. On the X-axis are dilutions of itraconazole. Fungal Growth is noted by a + and lack of growth by a −.

One can also see that the two compounds combine without antagonism in their antifungal activity. This alone is significant since sodium chlorite is a strong oxidizing agent and quite reactive to a number of organic compounds. When used together, their effect is combined to yield growth inhibition as noted by the data point circled in red. At those concentrations fungal growth would have occurred if only one compound were present. Therefore, the present invention provides an improved antifungal effect over either compound alone.

The experiments were repeated with a different strain, Trichophyton mentagrophytes ATCC MYA 4439. Again a significant combined antifungal activity was shown to be superior to either compound alone.

For T rubrum, ATCC MYA 4438, Itraconazole was decreased at least 2-fold by dropping from a required amount of 0.06 ug/ml to <0.03 ug/ml.

For ATCC MYA 4439, the amount of Itraconazole required to inhibit the fungus was decreased at least 4-fold from 0.125 ug/ml to <0.03 ug/ml.

These studies indicate that when sodium chlorite is combined with antifungals in the azole group, lower concentrations of sodium chlorite may be used to create an effective antimycotic agent, which is an improvement over previous sodium chlorite containing compositions and previous azole group antifungals. For this purpose, concentrations of sodium chlorite from around 0.015% are effective.

When azoles in concentrations from 0.03% to 15%, preferably 1-3%, most preferably 2% are combined with sodium chlorite in concentrations from around 0.015- 3%, positive results can be observed against the dermataphytes responsible for onychomycosis in broth culture and in vivo tests. At higher concentrations from 0.75 to around 3.5%, preferably around 2%, sodium chlorite penetrates the nail and skin without irritation acting as a carrier for the antifungal agent. Sodium chlorite is also effective alone against dermataphytes that cause tinea pedis and onychomycosis in broth culture and in vivo at concentrations ranging from 0.25-4%.

Discoloration characteristic of onychomycosis clears progressively to a more normal color along the entire length of the nail. There is no need to wait for the nail to grow out to see results. The texture and consistency of the keratin debris begins to soften and change from self-adhering and difficult to remove to a flaky substance that can be removed at home with simple manicure tools at home.

In vivo tests further demonstrate that when peroxides, specifically but not limited to hydrogen peroxide or benzoyl peroxide are combined with sodium chlorite, the beneficial results include quick improvement of nail appearance preferably when combined with antifungals, either azoles or allylamines. Beneficial results have also been noted against bacterial and fungal infections of the skin. When hydrogen peroxide or benzoyl peroxide is used with a chlorite salt, an Allylamine such as Terbinafine may be used successfully according to this invention.

Regarding pharmacological stability of the composition, Patent application 20040037891 (included in its entirety) describes experiments to determine pharmacological stability of combinations of sodium chlorite and hydrogen peroxide. Stability of the combined components were established at a pH range. of 6.0-8.8 through the use of a spectrophotometer (e.g., Lambda 20 Model UV—Vis. Spectrophotometer) to find and measure the levels of sodium chlorite and the generation of chlorine dioxide at varying pH levels.”

As indicated earlier, the chlorite/peroxide preparation of the present invention, whether it be in the form of liquid solution, gel, ointment, cream, spray, etc., is specifically composed to maintain chlorite such as sodium chlorite and hydrogen peroxide as active ingredients at a pH range of 6.0-8.8 without generating chlorine dioxide during storage at room temperature.”

The stability of the antifungal agent during storage also needs to be taken into consideration. For example, itraconazole is known to degrade under certain storage conditions unless precautions or stabilizers are used. Int J Pharm. 2011 Jul 29;414(1-2).

When sodium chlorite at pH 7 (which does not create chlorine dioxide) in concentrations from 0.5-2.0% were tested against Candidas albicans, 100% kills were observed without later growth upon removal of the sodium chlorite. This implies that not only vegetative growth was killed but also any spores were inactivated. The lower end of fungal killing and even lower concentration needed for fungal inhibition are much lower. When combined with conventional antifungal agents, chlorite ions have been shown to inhibit fungi at even a lower concentration. See FIGS. 1 and 2.

At 0.5%, toxicity against human cells is low and toxicity against Candidas albicans was 100% making ranges around 0.5% the desirable blood concentration with the other ranges provided above acceptable ranges. These concentrations are attainable by oral, injection, infusion and application to mucous membranes. Side by side comparisions with standard antifungal agents gave significantly superior results compared to fluconazole against which it was tested in vitro. Thus, sodium chlorite alone, at pH levels that do not create chlorine dioxide, is effective alone or in combination with antifungals against Candidas albicans.

Broth culture tests also indicate that when combined with azoles against more than one fungus, significant increased antifungal activity has been observed both by enhancing anti-fungal effect of the chlorite salt and simultaneously increasing the effect of the azole (in the case of Trichophyton rubrum, the MIC of itraconazole dropped fourfold. In the case of Trichophyton Mentagrophytes two-fold. Further, it has been demonstrated in broth culture tests that when combined with itraconazole against Trichophyton rubrum sodium chlorite's MIC dropped eightfold.

Sodium chlorite both alone and in combination with conventional antifungal agents is a superior antifungal agent compared to the antifungal alone. Sodium chlorite may be used alone or in combination with other antifungal agents against a variety of fungi infecting humans including but not limited to Absidia corymbifera, Ajellomyces capsulatus, Ajellomyces dermatitidis, Arthroderma benhamiae, Arthroderma fulvum, Arthroderma gypseum, Arthroderma incurvatum, Arthroderma otae, Arthroderma vanbreuseghemii, Aspergillus flavus, Aspergillus fumigatus, Aspergillus niger, Blastomyces dermatitidis, Candida albicans, Candida glabrata, Candida guilliermondii, Candida krusei, Candida parapsilosis, Candida tropicalis,Candida pelliculosa, Cladophialophora carrionii, Coccidioides immitis, Cryptococcus neoformans, Cunninghamella sp., Epidermophyton floccosum, Exophiala dermatitidis, Filobasidiella neoformans, Fonsecaea pedrosoi, Fusarium solani, Geotrichum candidum, Histoplasma capsulatum, Hortaea werneckii, Issatschenkia orientalis, Genus Madurella; Madurella grisae, Malassezia furfur, Malassezia globosa, Malassezia obtusa, Malassezia pachydermatis, Malassezia restricta, Malassezia slooffiae, Malassezia sympodialis, Microsporum canis, Microsporum fulvum , Microsporum gypseum, Mucor circinelloides, Nectria haematococca, Paecilomyces variotii, Paracoccidioides brasiliensis, Penicillium marneffei, Pichia anomala, Pichia guilliermondii, Pneumocystis carinii, Pseudallescheria boydii, Rhizopus oryzae, Rhodotorula rubra, Scedosporium apiospermum, Schizophyllum commune, Trichophyton mentagrophytes,Trichophyton rubrum, Trichophyton verrucosum, Trichophyton violaceum, Trichosporon asahii, Trichosporon cutaneum, Trichosporon inkin, Trichosporon mucoides.

The same and other fungi also infect animals and may be treated in the same manner with the same composition. A number of different fungi infect plants, particularly those of the Ascomycetes and Basidiomycetes. A pesticide may be prepared from a metal chlorite and applied to the plant for treating both surface and systemic infections.

A pattern observed indicates that sodium chlorite alone at a pH that does not create chlorine dioxide and in combination with conventional antifungal agents is a superior antifungal agent than the antifungal alone against 100% of fungi tested. Routine testing will determine best concentrations of sodium chlorite alone or in combination with conventional antifungal agents against variety of fungal diseases including but not limited to Allergic bronchopulmonary aspergillosis, Aspergilloma, Aspergillosis, Athlete's foot, Basidiobolomycosis, Basidiobolus ranarum, Black piedra, Blastomycosis, Candidiasis, Chronic pulmonary aspergillosis, Chytridiomycosis, Coccidioidomycosis, Conidiobolomycosis, Covered smut (barley), Cryptococcosis, Cryptococcus gattii, Dermatophyte, Dermatophytid, Dermatophytosis, Dimorphic fungi, Endothrix, Entomopathogenic fungus, Epizootic lymphangitis, Esophageal candidiasis, Exothrix, Fungemia, Histoplasmosis, Massospora cicadina, Template:Mycoses, Mycosis, Mycosphaerella fragariae, Myringomycosis, Oral candidiasis, Paracoccidioidomycosis, Pathogenic fungi, Piedra, Piedraia, Pneumocystis pneumonia, Sirococcus clavigignenti-juglandacearum, Sporotrichosis, Thousand cankers disease, Tinea, Tinea barbae, Tinea capitis, Tinea corporis, Tinea cruris, Tinea faciei, Tinea incognito, Tinea nigra, Tinea versicolor, White nose syndrome, Zeaspora, Zygomycosis.

For diseases of the skin, there exists a need for a treatment option that combines sodium chlorite's penetrating action together with effective antifungal agents in a one step single container. One embodiment of the present invention is a one-step topical lotion that treats onychomycosis that is pharmacologically stable. Because of its slightly alkaline pH and antibacterial properties, Clotrimazole is a preferred candidate for formulation into a one step treatment with sodium chlorite though any other azole may be used.

In another method of this invention, miconazole nitrate was tested for topical treatment of the symptoms of onychomycosis. Because of its high acidity, miconazole nitrate (formulated with benzoic acid, BHA, mineral oil, peglicol 5 oleate, pegoxol 7 stearate, and purified water resulting in a pH of 3) may react with sodium chlorite and water solution to create chlorine dioxide. It is an extremely effective combination but is less pharmacologically stable thus requiring two containers, a twin dispenser or other means to keep the reactive components separate. A formulation with miconazole nitrate at a safe and effective concentration at a pH between 6-8.8 would also be suitable for a one step formulation.

The oil base of the 6.5% tioconazole tested compromised its effectiveness as a treatment option for use on the nail. The oil component of the tested formulation of tioconazole appeared to bloat the nail and discolor it while under treatment. Though tioconazole 6.5% in an oil base was temporarily displeasing aesthetically, it was notably effective against onychomycosis. It is further surmised that safe concentrations of imidazoles above 1-2% such as the 6.5% concentration tested in tioconazole seem to be preferable, though not required for the most effective results. If daily use of such concentrations may not be advisable because of toxicology concerns, higher concentrations of antifungals may be administered at the initial stage of treatment, and then periodically applied on a 1-7 day rotation or a weekly rotation basis. For example, higher concentrations may be used in bursts of 1-7 days followed by treatment at lower concentrations of 1-7 days. Such a regimen pattern could be repeated as necessary until symptoms clear.

Ketaconazole was tested with excellent results when combined with sodium chlorite against fungal infections of the skin and nails.

A preferred method of the aforementioned embodiments combines a cream, lotion or gel base containing 0.5-15%, preferably around 1-2% of a topical antifungal agent or topical antibiotic agent with sodium chlorite, preferably above 1%, more preferably about 2%. This composition may be used to treat dermatological conditions such as, but not limited to nail fungus and bacterial and fungal infections of the skin. A pH between 6-8.8 is preferred. Another most preferred embodiment of the present invention adds 0.005% -5%, preferably about 3% peroxide, preferably but not limited to hydrogen peroxide or benzoyl peroxide with at a preferable ratio of 1 part peroxide containing composition to between 2 parts to 10 parts of the resulting cream, lotion or gel containing sodium chlorite composition. This composition may be used to treat dermatological conditions such as, but not limited to nail fungus and bacterial and fungal infections of the skin such as acne. Also, while corns and bunions are usually not considered infections, a fungal component may be present. Improvement in the size and appearance of corns and bunions in the area of treatment was noticed by test patients during the course of treatment. Therefore, for the purposes of this specification, corns and bunions are considered to be included under skin infections.

Another embodiment of the present invention adds about 0.005% -5%, preferably about 3% peroxide, preferably but not limited to hydrogen peroxide or benzoyl peroxide, to a sodium chlorite composition. Preferred ratios of peroxide to sodium chlorite are 1 part peroxide to between 2 parts to 10 parts of the resulting cream, lotion or gel containing sodium chlorite.

Another preferred method of the aforementioned embodiments is a non-toxic but effective anti-fungal amount of sodium chlorite in oral form such as a capsule or tablet; a parental form such as an injection, intravenously administered solution or infusion to other part of the body, or a transdermal or transmucosal administration. This composition can be used to treat systemic disease or diseases of internal organs. The dosage will vary with the patient and severity of the infection. Suitable dosages are those needed to provide a systemic concentration shown to be effective in vitro without significant toxicity. For example, systemic concentrations from about 0.01 micrograms per milliliter up to about 0.5% may be obtained by administering sufficient sodium (or other metal ion) chlorite to achieve such concentrations. The chlorite salt may be administered alone or in combination with other antifungal agents in one or more compositions.

Another preferred method of the aforementioned embodiments is a non-toxic and effective amount of sodium chlorite in the form of a suppository, cream or gel or solution or an effective concentration of sodium chlorite to form a vaginal rinse. This composition can be used to treat diseases of the reproductive system.

For topical treatments, it is advantageous to include an oil during the treatment. The oil may be intergral to the topical composition containing active ingredients or it may be added afterwards as a protective coating and/or to provide additional effects or to counter the harshness of the active chemicals. Examples include olive oil along with chlorite and an optional antifungal for treating candidia vaginal infections and the like. The treatment may also be used to treat any other topical infections such as ringworm and onychomycosis

Another preferred method of the present invention includes the use of sodium chlorite alone. Sodium chlorite alone is effective against dermataphytes that cause Tinea Pedis (Trichophyton mentagrophytes) and onychomycosis (Trichophyton rebrum) in broth culture at low concentrations. Agar plate tests show that sodium chlorite alone is effective against Candida albicans.At 0.5%, it kills Candida albicans rather than simply inhibiting its growth. It can be concluded that sodium chlorite alone in concentrations significantly below that may be effective against a variety of fungus.

Another advantage to the present invention is that when stable sodium chlorite that does not form chlorine dioxide is used alone and in combinations described herein, the chemical compound(s) are superior against pathogenic fungi than currently recognized antifungal agents alone.

One advantage of the present invention is that it is safer than current conventional antifungal oral medications in that it does not harm the user's liver. Furthermore, the interaction between certain antifungal medications inhibiting the P450 complex and other medications are known problems. The use of sodium chlorite avoids such problems.

Yet another advantage of the present invention is that the preferred combinations are likely to be pharmacologically stable at recommended pH levels for eighteen months. Even with off-gassing of chlorine dioxide, a number of mouthwashes and other oral hygene products are sufficiently shelf stable in dry form. The present invention, using a higher pH and less or no release of chlorine dioxide is even more stable.

Yet another advantage of the present invention is that the preferred formulations do not irritate either the nail or the skin surrounding the nail when used at pharmacologically effective concentrations.

Yet another advantage of the present invention is that the method penetrates the surface of the nail, where other topical treatments cannot, thereby effectively attacking the fungus located under the nail.

Another advantage of this treatment is that the biofilm forming a protective-like coating caused by fungus at the nail's distal edge becomes penetrable and quickly improves in appearance upon treatment with chlorite solutions. Fungus impregnated keratin debris changes into a slightly crumbly removable substance.

Yet another advantage of the present invention is that it does not degrade the nail, but restores it to a healthy consistency and appearance when chlorite is used alone or with antifungals.

Yet another advantage of this invention is that debridement of the nail surface is not required or desired for effectiveness. Thickened nails may be “thinned” from beneath the distal edge with common nail tools (though this is not required) for improved nail appearance. The nail is not weakened by the treatment but restored.

Yet another advantage of the method of treating onychomycosis according to the present invention is that it creates perceivable improvement to the nail as early as after several days to one month after start of treatment, where such results do not depend on the nail growing out.

Another advantage is that the nail appears to grow out clear of fungal discoloration while existing discolored nail restores to a normal color.

Yet another advantage of the method of treating onychomycosis according to the present invention is that discernible results can be seen within one to two weeks even with heavily infected toenails. Presently, the effectiveness of currently marketed topical products is limited to light infections that are confined to the distal edge of the nail.

Yet another advantage of this invention is that strict compliance is not necessary for good results. Though twice daily treatment is recommeded, good results have been obtained with once daily treatment. Less frequent treatment is possible depending on the amount of active ingredient(s) present, the type and extent of the infection, the use of delayed release of the pharmaceutically active ingredient(s) and/or antievaporative agents (such as an oil). When used as a maintence treatment or occasional treatment when suspected of being exposed to fungi, the treatment schedule may be even less frequent or as needed.

Yet another advantage of this invention is that the results are cosmetically pleasing. Hard brittle nails soften to a more normal consistency through the use of this invention. Discoloration changes to a normal colour. Cracks and gouges appear to fill in. Flaking nails caused by onychomycosis begin to heal within two-three weeks.

Another advantage to the present invention is that alone and in combinations described herein, the chemical compound(s) are superior against pathogenic fungi than current antifungal agents alone.

Still another advantage to the present invention is the killing of fungi rather than simply inhibiting its growth. Internal and systemic infections frequently occur in immunocompromised individuals who are already taking other medications. Without an effective immune system, fungistatic treatments would need to be continued for a long duration. Since many conventional antifungal medications have side effects and interactions with other medications, it would be beneficial to either avoid such treatments or to limit their duration. The present invention using chlorite both avoids drug interactions and if used in combination with conventional antifungals, the duration of treatment may be shortened considerably.

### Dosage and Administration

For dermatological conditions, the treatment is topically applied. The objective is to cover the entire affected area. In the case of onychomycosis, the entire nail, under the nail and cuticle and affected periungal tissue. Since covering rough skin and nails uses more of the compositions than healthier nails, a specific amount of the composition is not detailed here. It is advisable, though not necessary, to gently rub blended composition(s) into the nail. It is also recommended that an individual leave a final layer of the composition on the affected area until bathing or the next treatment. It will sink into the nail and skin leaving little trace.

In one variation, a composition containing sodium chlorite and an antifungal is administered to an infected nail at least once daily, preferably at least twice daily, for the duration of the infection, which may be from 1-2 or 1-12 months or more. In some instances, an individual will continue treatment with the composition for any period of time after the infection is eradicated, as a measure to prevent re-infection or relapse. In some instances, an individual with no current signs of fungus-infected nails will apply the composition prophylacticly after a nail injury or possible exposure to the dermataphytes that cause onychomycosis. Less than once daily dosing regimens are contemplated with sodium chlorite and antibiotic/antifungal compositions, preferably azole class antifungals comprising from about 10 weight percent azole to about 15 weight percent azole. Even at standard concentrations of antifungals, effectiveness of treatment has been maintained even when dosing regimens are somewhat erratic. For example, treatment may occasionally be less than once daily. Mixed dosing regimens are also provided. It has been demonstrated that cycling through different antifungals is effective. For example, two weeks of treatment with 2% ketaconazole and sodium chlorite is followed by 2% miconazole nitrate and sodium chlorite, or an antifungal with a different mechanism of action such as terbinafine.

Another preferred method of the aforementioned embodiments combines a solution, cream, gel, or lotion consisting of around 0.005% -5%, preferably 3% peroxide, preferably but not limited to hydrogen peroxide and a 0.5% -3% sodium chlorite and water solution, cream gel or lotion at a ratio of 1 part peroxide to 2 parts sodium chlorite solution. 1:1-1:8 ratios of peroxide to sodium chlorite solution/have been used effectively. This composition may be used alone or with antibiotics or other beneficial agents to increase their penetration to aid treatment of bacterial and fungal infections of the skin and nails. A pH of around 6-8.8, preferably around 6 is preferred. A two-step product or twin barrel dispenser may be ideal.

A solution or cream, or gel, or lotion with 0.1-10%, preferably about 0.5 to about 5%, most preferably around 2% sodium chlorite may also be created for use with antifungal, antibiotic or other beneficial agents to increase their penetration thereby enhancing the treatment of diverse bacterial and fungal infections of the skin and nails. A pH of around 6 -8.8, preferably above 6 is preferred.

Beneficial agents that promote healing and the general health of nails may be augmented by the above described basic penetrating compositions. A non-limiting list includes products that moisturize, exfoliate or that treat otherwise healthy nails and that peel, flake or are dry or brittle. For example, when 1 part 0.75-2% sodium chlorite is combined with around 1 part 2-10% alpha hydroxyl or lactic acid, preferably glycolic acid, preferably around 7% (for home treatment) and/or 2-10% peroxide such as, but not limited to benzoyl peroxide, an excellent cuticle treatment that may also be used to soften and exfoliate periungal tissue is achieved. The final product has a pH of 3.5 or greater. After treatment where product is left on from 3 minutes to several hours, cuticle can be pushed back and then dead cuticle is removed by gentle rubbing with a bath towel or textured disposable cloth. When standard or “micro” “cleansing grains” are added, an effective exfolliant for skin of the entire face and body; not just skin of the foot is achieved.

There exists a need for a nail polish that covers up onychomycosis while treating it. Another embodiment of this invention is an onychomycosis treatment that may be used with nail polish. It has been discovered that when sodium chlorite is combined with an azole class antifungal, then rubbed onto and under a polished nail, preferably one using nail polish containing water and pentavitin at least twice a day without changing nail polish; that the nail's appearance improves and signs of onychomycosis are reduced. Pentavitin is a unique water magnet molecule that banks moisture within the surface of the natural nail.

In another embodiment of an onychomycosis treatment that may be used with nail polish, when sodium chlorite and a peroxide, preferably but not limited to benzoyl peroxide, most preferably hydrogen peroxide are mixed at a ratio ranging from 1:1 to 1:6, then combined with an antifungal, then rubbed onto and under a polished nail, preferably one using nail polish containing water and pentavitin at least twice a day without changing nail polish, the nail's appearance improves and signs of onychomycosis are reduced. For all treatments using nail polish, it is recommended that nails are also treated with the above described compositions before initial application of polish.

Azole class antifungals, such as clotrimazole, resolve Athlete's foot within 3 days to one week when they are combined with penetrating sodium chlorite above 0.1%, preferably around 2%. Itching and irritation are soothed within minutes of initial application. Note the in vitro tests featured in FIG. 2 that show a fourfold decrease in the MIC of itraconazole when used with sodium chlorite against Trichophyton mentagrophytes. At present imidazoles take at least two weeks of twice daily treatment for successful treatment of athlete's foot. This in vivo data is confirmed by the in vitro MIC tests against Trichophyton mentagrophytes graphed in FIG. 2. Preliminary testing confirms that up to around 2% sodium chlorite is tolerated in mucous membranes. According to preliminary data, sodium chlorite at concentrations between 0.25 -2% will likely augment conventional antifungal based treatment of thrush of the mouth and candidiasis of the vagina and tineas by enhancing penetration of active ingredients thereby speeding results and curtailing treatment time.

A preferred component of the present invention is the use of a chlorite salt. While the sodium salt was exemplified, other salts may be used such as potassium, calcium, magnesium, ammonium, and other salts. Concentrations in the range of about 0.01% to about 5% are preferred. Concentrations above 1% and below 3.5% are particularly preferred. Concentrations above 1.80% and below 2.12% are most preferred. It is preferred for the pH to be above six so that a stable shelf life can be maintained, though lower pH is acceptable. A lower pH that maintains a small amount of metal chlorite to be converted into chlorine dioxide may be used in the present invention when it functions to generate chlorine dioxide under acidic conditions and/or to maintain saturated chlorine dioxide conditions.

Another preferred component of the present invention is the use of azole antifungals which include include but are not limited to butoconazole, clotrimazole, econazole, miconazole, ketoconazole, terconazole, tioconazole, lanoconazole, luliconazole, bifonazole, clomidazole, croconazole, eberconazole, econazole, fenticonazole, flutimazole, isoconazole, neticonazole, omoconazole, oxiconazole, setraconazole, sulconazole, fluconazolen and itraconazole.

Most imidazole class antifungals are sold over the counter in the USA and have high safety profiles. Concentrations of the imidazole class according to this invention may range from 0.01-15%, preferably from 1-3%.

A preferred imidazole antifungal of the present invention is clotrimazole. It has antibiotic as well as antifungal properties. At present, clotrimazole is used to treat yeast and fungal infections (including candidiasis and tinea) of the vagina, and skin such as athlete's foot, jock itch, and body ringworm.

Another preferred imidazole antifungal agent is miconazole nitrate, especially when used in conjunction with a gel or nail polish containing water and pentavitin.

Another preferred component of the present invention are antifungal agents which include, but are not limited to terbinafine, natrifine, amorolfine, polyene antifungals such as amphotericin B, nystatin, etc., natamaycin, naftifine hydrochloride, flucytosine, griseofulvin, potassium iodide, butenafine, ciclopirox, ciloquinol (iodochlorhydroxyquin), haloprogin, tolnaftate, aluminum chloride, potassium permanganate, selenium sulphide, salicylic acid, zinc pyruthione, bromochlorsalicylanilide, methylrosaniline, tribromometacresol, undecylenic acid, polynoxylin, 2-(4-chlorphenoxy)-ethanol, chlorophensesin, ticlatone, sulbentine, ethyl hydroxybenzoate, dimazole, tolciclate, sulphacetamide, benzoic acid and pharmaceutically acceptable salts thereof. These antifungals are more preferably used with at least one peroxide.

Another preferred agent is Pentavitin which is a unique water magnet molecule that banks moisture within the surface of the natural nail.

Alpha hydroxy acids are well known for use in the skin treatment and are a preferred component of the present invention. Representative alpha hydroxy acids include mandelic acid, lactic acid, glycolic acid, etc. and salts thereof. One or more of these (or their salts) may also be used to serve as a pH adjusting agent or a buffering agent. While citric acid/citrate buffer is typically used for buffering in an acidic range, other pH-adjusting agents or buffering agents may be used alone or in combination with the alpha hydroxy acids of the present invention. In order to buffer in a neutral to alkaline range, different buffers are preferred. Concentrations in the range of about 0.1% to about 20% are preferred. Depending upon the specific composition used and/or the intended frequency of use, narrower ranges are preferred. More preferred are ranges such as 0.2%-7% glycolic acid for daily use. Alpha hydroxy acids, particularly glycolic acids are particularly effective for cuticle treatments when combined with above 2% sodium chlorite.

Carboxylic acids such as, but not limited to benzoic acid may be used in this invention. Benzoic acid is used in common formulations of imidazole based antifungals such as miconazole nitrate.

Salicylic acid may also be used in the present invention. Concentrations in the range of 0.2 to 20% may be used, preferably 0.5 to 8%, more preferably 1 to 5%. Other organic acids may be used, especially those which are known pealing agents such as trichloroacetic acid.

A broad-spectrum antibiotic or combination of antibiotics may be administered topically or administered systemically. Other topical or systemic antibiotics may be used that are active at inhibiting bacteria in the nail bed and surrounding areas. While antifungal agents may be included in the category of antibiotics, in the present invention these antibiotics may be better described as broad-spectrum anti-bacterial agents. Neosporin® (bacitracin, polymyxin, neomycin) is one such commonly used topical antibiotic. While fungi are the primary infecting agent in onychomycosis, considerable dead and damaged tissue are produced during the infection which provides suitable conditions for growth of other microorganisms which may metabolize or otherwise reduce the effectiveness of antifungal treatments.

In an embodiment, sodium chlorite compositions and antifungal agents may be applied directly to the infected nails, nail bed and surrounding skin. Sodium chlorite and antifungal agents may be applied to the skin nail or hair in any form, but it is preferred that the composition is in solution or emulsion form. Any chlorite salt that negatively affects the presence of conditions of the hair skin or nails such as onychomycosis of the finger and toenails may be used in the present invention. Any imidazole antifungal agent or similar antimycotic that preferably acts by breaking down the cell wall of a microorganism and/or leads to a buildup of hydrogen peroxide within its cells may be used in the present invention.

Other optional ingredients include the use of diluents, salts to make the compositions isotonic, emollients, humectants, pH buffers, beta hydroxy acids, thickeners, stabilizers, abrasives, anti-inflammatory agents and chemicals involved in generating chlorine dioxide such as a metal salt of hypochlorite. However this patent teaches that generating chlorine dioxide by such means is not necessary for a good result according to this invention and generally creates problems with shelf life. Higher alkalinity in sodium chlorite compositions generally appears to increase penetration of the nail and skin and enhance results.

The final form of one or more components to the composition of the present invention is preferably liquid, powder or spreadable solid (paste, gels, powders, ointments, integument, liquid or powder for inhalation under pressure to the lungs, gargle, douche, mouthwash, enema, etc.). A soap product containing some of the components of the composition of the present invention may be formed. This soap may be either in liquid or solid bar form. Components that are incompatible or volatile may be provided separately such as in a dropper bottle. In such a way a person may wash and simultaneously treat infected nails or prophylacticly apply some or all of the components to the nails, and if necessary followed by application of one or fewer liquids to the nails. Optionally, one may formulate a treatment bar with a different solidifying agent that resembles a soap bar but without the active soap ingredient. One example would be with the use of stearyl alcohol as the solidifying agent.

Compositions used in the present invention include those in one container and those prepared from multiple containers. The nature of some of the chemicals used makes them react with each other over time resulting in a product, which lacks one-year (or greater) shelf stability. In such a situation, the present invention may be a kit containing two or more containers of chemicals such that chemicals reactive with each other are kept in separate containers. Compositions that are stable together are preferably premixed. The contents of the containers may be mixed immediately prior to use, added simultaneously or sequentially at the target site of infection or may be mixed together ahead of time and briefly stored before use. For example, some components may be mixed in liquid form and stored for example, about two months or so whereas the original components may be shelf stable for over one year. Storage under refrigeration or freezing conditions may extend the acceptable storage time. The final kit may include packaging for the two or more containers and instructions for preparation and use. Individual containers may contain any form of solids, liquids, gels, ointments, creams, roll-on, aerosol sprays, nail polish etc. At least one of the containers or an additional empty container may be re-sealable after dispensing its contents after each application. One or both of the containers may contain a dry ingredient which is inactive until activated by water or a component in a different container. In such a situation, the concentrations and amounts would be that needed to produce the amounts or concentrations mentioned at the site on/in the body or item.

More specifically, one may use a two (or more) barrel syringe or other multi-chambered container to co-extrude the different composition components which are either mixed immediately before applying to the nail or are applied simultaneously or sequentially on the nail.

An alternative container may be a multipack containing individual dosages of one or more of the containers; for example a bottle containing capsules or pouches (or other individual dose container) where each capsule contains one or more components of the composition of the present invention. Different bottles may contain different capsules and different capsules may be opened immediately before use.

The components may also be held in the form of an emulsion with the active ingredients in one or more discontinuous phases in the emulsion. Water-in-oil emulsions are particularly preferred and are well known per se. This allows reactive components to be kept apart until mixed and applied. Furthermore, judicious selection of the continuous phase and optionally a surfactant may entrap volatile or reactive compounds away from each other leading to a longer shelf life. In a preferred embodiments, the oil phase comprises 30-90 vol % of the oil-in-water emulsion, more preferably 50-80%. Furthermore, while the present invention is not limited by the nature of the surfactant, in some preferred embodiments, the surfactant is a polysorbate surfactant (e.g., TWEEN 20, TWEEN 40, TWEEN 60, and TWEEN 80), a pheoxypolyethoxyethanol (e.g., TRITON X-100, X-301, X-165, X-102, and X-200, and TYLOXAPOL) or sodium dodecyl sulfate.

Alternatively, one may include an oil as a separate reagent after the active ingredient(s) have been applied topically. The oil is not significantly miscible in the aqueous reagent(s) containing active ingredient(s). The oil acts to reduce evaporation and oxidation by air as well as maintaining the active ingredient(s) on the body surface for a longer period of time. The oil may be inert or by itself contain pharmaceutical properties, such as ozonated sunflower oil.

Other compositions that release peroxide when ozonized or oxidized and is relatively non-toxic to humans may also be used in the present invention, either alone or in combination with one or more active ingredient listed above. Such a composition need not have oil-like properties such as honey. However, oils and other substances that release peroxides and other antimicrobial agents upon contact with chlorite are particularly preferred.

The oil may also impart a fragrance or counter the fragrance of sodium chlorite solutions. The use of an oil may ameliorate the harshness of the active ingredient(s), particularly sodium chlorite. Other desirable properties in the oil include defoliant properties, and hydrating/moisturizing, emollient or astringent properties. For topical treatment at certain locations, these may be significant properties for a product.

To enhance the activity of the oil with other active ingredients, the oil may be pre-treated with ozone. The ozonation reaction is more preferred when the oil is unsaturated.

Examples of oils include essential oils, many of which are known to have antifungal activity by themselves. Examples include bitter almond oil, cedarwood oil, cinnamon oil, citronella oil, clove oil, coriander oil, eucalyptus oil, frankincense oil, helicrysum oil, juniper oil, lavender oil, manuka oil , mustard oil , myrrh oil, orange and palm and rosa oils, olive oil, oregano oil, parsley oil, patchouli oil, pennyroyal oil, ravensara oil, rue oil, sage oil, tansy oil, tea tree oil, or white birch bark oil.

Plant oils may also be used which have little or no antifungal activity. Examples include: include flax oil or linseed oil, safflower oil, hemp oil, evening primrose oil, sunflower oil, chia oil, perilla oil, walnut oil, candle nut, soybean oil, corn oil, coconut oil, cottonseed oil, squalene oil, rapeseed oil, wheat germ oil, canola oil, sesame oil, cotton seed oil, rice bran oil, beechnut oil, sweet almond oil, avocado oil, theobroma oil, jojoba oil, peanut oil, castor oil and grape seed oils/extracts. However, some of these oils give off a disagreeable smell when oxidized such as sunflower.

Animal oils, frequently called fats, may also be used, these include lanolin, fish oil, butters (generally clarified), egg yolk extracts and oil extracts of the waste products of slaughterhouses and rendering facilities. Mineral oils, particularly such as straight, branched and cyclic hydrocarbons may also be used. Either refined or synthetic mineral oils may be used. Aromatic hydrocarbons may be used but parafinnic mineral oils are preferred.

The oil may be in a separate container or in one of the containers containing an aqueous liquid with active ingredients. If in the same container, the oil and aqueous phases are primarily separate being in the form of two clear separate phases or in an emulsion, preferably a water-in-oil emulsion. A surfactant or emulsifier may be present.

It is another embodiment of the present invention that a topical composition containing at least olive oil (with or without ozonation) and sodium chlorite may be used as a treatment after traumatic injury. A conventional antibacterial agent (e.g. neomycin) may be beneficial in the composition. While the examplefied use is to treat fissures and cracks in the skin, such a composition would also function with antibiotic and healing effects. This also serves to prevent infection from any existing fungal infection elsewhere on the body.

Sodium chlorite compositions and antifungal agents may be applied directly to the infected nails, nail bed and surrounding skin, mucus membranes or skin topically. It may also be administered systemically by oral, transdermal, trans mucus membrane or parentally by injection or infusion in effective concentrations. Sodium chlorite and antifungal agents may be applied to the skin nail or hair in any form, but it is preferred that the composition is in solution or emulsion form. Any chlorite salt that negatively affects the presence of conditions of the hair, skin or nails such as onychomycosis of the finger and toenails may be used in the present invention. Any antifungal agent, but preferably any azole antifungal agent or similar antimycotic that preferably acts by breaking down the cell wall of a microorganism and/or leads to a buildup of hydrogen peroxide within its cells may be used in the present invention. One may conclude that nail penetration may be responsible for the dramatically increased efficacy represented by the experiments below since 20-40% improvement with the combined compositions cannot account for the in vivo results which would be quantified in much higher ranges—even as high as 78-92% in some instances.

At the beginning of treatment of extremely infected nails, double or sometimes triple portions of antifungals, antibiotics and benzoyl peroxide in the recommended ratios may be required to cover rough skin and nails.

### Composition of Tested Treatments

Unless otherwise noted, compositions are combined 1:1, mixed in hand or applied separately directly to the nail unless packaged pre-mixed as in Composition D and D1. The objective is to cover the entire nail, under the nail, cuticle and affected periungal tissue. Since covering rough skin and nails uses more of the compositions than healthier nails, a specific amount of the composition is not detailed here; instead we use ratios. It was advised to gently rub the blended compositions into the nail. It was also recommended that the subject leave a final layer of the combined composition on the nail until bathing or the next treatment. The compositions were adsorbed into the nail and skin leaving little trace within twenty minutes. The Compositions have been used up to six times a day though use at least twice a day was recommended. Good results have been obtained with less regular treatment. Treatment may be continued until nail grows out clear, approximately one year. Repeat use may be required to maintain healthy nail appearance.

The terms “CS” and “CS solution” indicates a solution created with sodium chlorite and water. The pH was adjusted to 5-9, preferably between 6-8 using either citric acid or glycolic acid. Other alpha hydroxyl acids and any other substance that adjusts pH down without dissipating SC before its use are acceptable for this purpose. 0.01-0.03% ribose, preferably 0.001% ribose may be added to the solution.

Prior to the application of the compositions involving the sodium chlorite solution, one to two drops of the sodium chlorite solution may be applied to the surface, cuticle and beneath each individual affected nail preparatory to application of antifungal cream or antifungal cream/sodium chlorite compositions.

Antifungal formulations may contain an allymine, amorolfine (or amorolfin). Amorolfine has previously been used for the treatment of onychomycosis, and it is similar to the allylamine Terbinafine. This antifungal was shown effective with sodium chlorite above 1%, preferably 2% and a peroxide preferably benzoyl peroxide or hydrogen peroxide to treat fungal conditions of the skin and nails. The sodium chlorite at the aforementioned concentrations penetrates the nail and prevent relapse. While amorolfine, (marketed as Curanail, Loceryl, Locetar, and Odenil), does not penetrate the nail by itself.

### BRIEF DESCRIPTION OF THE DRAWINGS

FIG. 1 is a graph illustrating Minimum Inhibitory Concentrations Study (mic) results combining sodium chlorite and itraconazole against Trichophyton rubrum. Results indicate a combined effect.

FIG. 2 is a graph illustrating Minimum Inhibitory Concentrations Study (mic) results combining sodium chlorite and itraconazole against Trichophyton mentagrophytes. Results indicate a combined effect.

FIG. 3 is a graph illustrating Minimum Inhibitory Concentrations Study (mic) results combining sodium chlorite and fluconizole against Candidia albicans.Results indicate a combined effect.

FIG. 4 is a series of photographs of a patient taken during the treatment.

FIG. 5 is a series of photographs of a patient taken during the treatment.

FIG. 6 is a series of photographs of a patient taken during the treatment.

Note on Photographic Documentation: The effectiveness of this treatment can be determined by changes in the appearance of the nail because the translucency of the nail. It is noted that the treated new nail appears to grow out clear.

For purposes of this invention, R1 describes the right great toe, counting from R1, R2, R3, R4, to the “little toe” described as R5. L1 is the great toe of the left foot with ensuing digits numbered L1, L2, L3, L4, to L5, which represents the “little toe”.

### EXAMPLES DEMONSTRATING THE EFFECTIVENESS OF CHLORITE SALTS

Example 1

Composition D was prepared by combining 15 g (2%) clotrimazole cream with 0.4 g (2%) sodium chlorite solution. pH was maintained at about 6.

Subject C had a moderate growth of fungus evidenced by yellowed discoloration of ⅓ of the right digit's toenail. A prominent lengthwise ridge ran across the nail and served as a demarcation point of the end of evident fungal infiltration of the nail. She also had a dark discoloration on her left digit one along with white vertical striations.

She applied the premixed chemical component by rubbing the cream into affected periungal tissue, her nail, cuticle and under the distal edge at least once a day, but no more than three times a day. She was instructed to administer it twice a day. Dark discoloration on right middle front of toe L1 faded with use. After 49 days, the nail appears normal on right 1^{st} toenail. Even though patient compliance was irregular her result was excellent.

Example 2

Subject R was diagnosed with onychomycosis. He had a severe growth of fungus evidenced by broken skin and discolored nails distended by keratin build up. Different compositions were applied to his nails. He treated nail R1 according to Example 1, Composition D for the first 22 days. Then he treated the nail for at least the following sixty days with Composition Dl.

Composition D1 was prepared by combining 2% clotrimazole cream with 2% sodium chlorite solution. pH was maintained above 6. The resulting creamy lotion was combined with 3% hydrogen peroxide at a ratio of one part peroxide to 3 parts lotion.

He applied the premixed chemical components by rubbing the cream into his nail, cuticle and under the distal edge at least once a day, no more than four times a day. Instructions were to apply it twice a day.

Composition D was effective. However Composition D1 lightened the persistent nail discoloration surprisingly quickly. Definition of the nail crescent becomes narrower. Nail continues to be treated according to Composition D1 and continues to improve.

Example 3

Tests were conducted with various combinations of compositions on 4 subjects.

### Components Used in Tests

Eighty percent sodium chlorite was used to create all sodium chlorite solutions and one step (Composition D, D1). All sodium chlorite containing formulations used herein were prepared by ClearCrescent Technologies. Glycolic acid used to adjust pH in the formulation of Composition D and used to construct sodium chlorite solutions was manufactured and distributed in Acros, N.J. Benzalkonium chloride was under the trademark, BenzaRid manufactured and distributed by Nature's Innovation Inc, Buford, Ga. 2% clotrimazole, 2% miconazole nitrate and 6.5% tioconazole ointment were commercial generic ointments. Terbinafine in the form of Lamisil AT Gel was distributed by Novartis Consumer Health, Inc. Benzoyl peroxide and glycolic acid were distributed by Guthy-Renker, Palm Beach, Calif. under the name of Proactiv Solution Repairing Lotion® and Proactiv Revitalizing Toner® respectively. Sodium chlorite was manufactured and distributed by the Ricca Chemical Company. Tested nail polishes include those containing Pentavitin and water manufactured by Prima Technologies in Nesconset, N.Y. Other tested polishes include Sally Hansen Double Duty Base and Top Coat ® manufactured and distributed by Dell Laboratories. ORLY French Manicure ® was manufactured and distributed by Orly International, Los Angeles, Calif. Glycolic acid 7% was used by dropper in the form of Proactive Revitalizing toner or in its pure chemical form to adjust ph.

Liquid was measured by drops from a standard pipette. A total of 1-3 drops were used atop and under each infected nail.

1 Part of a solid is defined as ribbon 0.5 cm wide and 0.5 cm long such as is extruded from a tube.

Unless otherwise noted, all measurements of different components range from 1:1, sodium chlorite: antifungal to 1:4 sodium chlorite: antifungal. Vagaries of self-dosing permit wide latitude. The intent is to use sufficient quantities of single or combined compositions to rub into, or to coat the entire affected nail and leave a rim of treatment compositions under the nail's edge.

### Abbreviations Defined

SC or sc, denotes sodium chlorite.

CS or cs denotes chlorite salts.

Clot or clot denotes clotrimazole.

BP or by denotes benzoyl peroxide.

BC denotes benzalkonium chloride

SC/P denotes sodium chlorite and peroxide solution

### Subjects Tested

### Subject R.

Background: Male Subject R was diagnosed with onychomycosis. He formerly used 3 step process detailed in patent application Ser. No. 12/587,495. After a positive result within the first two months of treatment, but with clear signs that fungus was still present, though visually much improved, subject R abandoned treatment for approximately 6-7 months. Nails relapsed. He asked for a simpler treatment regimen, and asked to resume treatment; ONE STEP (Composition D) was used.

Conclusion: R1-5 Good result with first 23 days of treatment according to Composition D.
R1 Excellent result with Composition D1. D1 treatment continues on all toes.
L1 treated according to COMPOSITION J (clotrimazole 2% alone) resulted in relapse.
L1 Treatment switched to D1 after 23 days. D1 treatment continues on all toes.

### Subject C

Background: Subject C is a 53 year old female. She estimates that she has had symptoms of Onychomycosis for 2-4 months.

Conclusion: Treated with Composition D; Excellent result. Treatment continues.

### Subject K:

56 year old female with severe KOH test diagnosed onychomycosis: Treated with various CS treatments according to patent application Ser. No. 12/587,495 with excellent progress before testing treatments included herein.

Conclusion: Relapse upon which testing was based triggered by use of Compositions A and B.

Good results according to Compositions D, D1, G, H, I, I1. Composition E was effective against fungus but aesthetically unacceptable.

Subject S: Male with KOH test diagnosed onychomycosis for more than one year.

Conclusion: Treated with Composition D. Good result. Treatment continues.

TABLE 1
TREATMENTS USED

| | Subject K | Subject C | Subject R | subject S |
|---|---|---|---|---|
| A | x | | | |
| B | x | | | |
| C | x | x | | |
| D | x | x | x | x |
| D1 | x | x | x | x |
| E | x | | | |
| F | x | | | |
| F 1 | x | | | |
| G | x | | | |
| H | x | | | |
| I | x | | | |
| I1. | x | | | |
| J | | | x | |
| K | x | | | |
| L | x | | | |
| M | x | | | |
| N | x | | | |
| O | x | | | |
| P | x | | | |

TABLE 2
A. ONE PART 1% benzalkonium chloride (BC) to ONE PART
benzoyl peroxide (BP) alone
B. ONE PART 1% benzalkonium chloride (BC) to ONE PART
6% benzoyl peroxide + 2% sodium chlorite solution.
C. One-THREE PARTS 2% clotrimazole to ONE PART 2%
sodium chlorite solution.
D. One Step: Topical cream prepared with 2% clotrimazole and
2% sodium chlorite and 0.01% ribose with a final pH around 6.
Dl. One Step: topical cream prepared with 2% clotrimazole and
2% sodium chlorite and 0.01% ribose with a final pH around 6.
Hydrogen peroxide was added to resulting lotion at a ratio of 1
part hydrogen peroxide to 2-3 parts lotion.
E. ONE PART-TWO parts 6.5% ticonazole to ONE PART 2%
sodium chlorite solution was mixed in the palm of the hand and
applied to affected areas.
F. ONE PART-THREE PARTS 2% miconazole nitrate to ONE
PART 2% sodium chlorite solution (pH around 6) was mixed in
hand then applied to nail.
F1 TWO PARTs 2% sodium chlorite solution (pH around 6) was
mixed with ONE PART 3% hydrogen peroxide then stored. Twice
a day, apply 1-2 drops of SC/P solution to affected nails. Mix in
hand ONE PART 2% miconazole nitrate with ONE PART
solution of sodium chlorite solution and peroxide then apply to
affected areas.
G. Nail polish containing Pentavitin and water used in conjunction
with Two PARTS clotrimazole 2%, ONE PART sodium chlorite
solution 2% and ONE PART glycolic acid approximately 6%.
H. Nail polish containing Pentavitin and water was used in
conjunction with One Step (Composition D) containing
clotrimazole 2%, sodium chlorite 2%. Method was the same as
in Example G, but One Step was substituted for Clot and glycolic
acid.
I. Nail polish containing Pentavitin and water used according to
example 2 in conjunction with ONE PART-THREE PARTS 2%
miconazole nitrate (pH 3) ONE PART 2% sodium chlorite
solution (pH 6).
I1 Nail polish containing Pentavitin and water applied then used
in conjunction with ONE PART 2% miconazole nitrate (pH 3)
and ONE PART 2% sodium chlorite solution mixed with 3%
hydrogen peroxide with 1 part peroxide to 2 parts sodium chlorite
solution.
J. Clotrimazole alone applied to cover nail, cuticle and under distal
edge.
K. Added ONE PART 2% CS solution to 1 tsp lemon juice on the
nail to remove nail polish discoloration. Continued treatment with
2% CS solution and 3% hydrogen peroxide combined at a ratio
range of 1:2 to 1:4, peroxide to sodium chlorite.
L. Conventional nail polish used in conjunction with 2 ONE-
THREE PARTS 2% miconazole nitrate and ONE-TWO PARTS
2% sodium chlorite solution.
M. 2% Sodium chlorite solution (pH about 6) was combined with
3% hydrogen peroxide at the following ratio: 2 part sodium
chlorite to 1 part hydrogen peroxide and applied to affected area.
N. Three PARTS 1% terbinafine gel combined in hand with ONE
PART of a solution containing 2 PARTS 2% sodium chlorite
solution and 1 PART 3% hydrogen peroxide solution. pH was
around 6. Resulting gel was applied to surface, cuticle and under
the distal edge of affected nails after 1-2 drops of SC/P solution
was applied by dropper to each affected nail.
O. THREE PARTS 2% ketaconazole to ONE PART 2% sodium
chlorite solution (pH around 6) was mixed in hand then applied to
nail after applying sufficient SC solution to cover top and
underside of the affected nail and surrounding periungal tissue.
P. TWO-THREE PARTS 2% miconazole nitrate to ONE PART
2.10% sodium chlorite solution (pH around 6) was mixed in
hand then applied to nail after applying sufficient SC solution to
cover top and underside of the affected nail and surrounding
periungal tissue.
Q. TWO-THREE PARTS 2% miconazole nitrate to ONE PART
2.0% sodium chlorite pH around 8 was mixed in hand then applied
to nail after applying sufficient SC solution to cover top and
underside of the affected nail and surrounding periungal tissue.

### Results of Tested Compositions

Unlike most onychomycosis treatments, one can receive notable cosmetic results within days of beginning treatment and good to excellent cosmetic results within 40 days or less of beginning treatment. Excellent cosmetic results include a return to the original color of the nail. Another unique short term benefit of these treatments is that pain from nails distended from packed keratin debris has been reported relieved in as little as two weeks. The nail appears to be quickly penetrated by the chlorite salts. Consequently, the height of the arching nail was reduced as fungus was attacked. This results in a more normal nail contour. To the person suffering from the symptoms of onychomycosis, this speedy relief is sought after, but previous to these discoveries, unavailable. For these reasons, according to the objectives of this treatment, 14-50 day results are significant. It was noted that the treated new nail appears to grow out clear.

TABLE 3
Comparison of Tested Alpha Hydroxy Acids and Chlorine
Dioxide in DAILY Regimen Including Antifungal and Antibiotic.

Fungus Quality
1-Fungus was reduced, was easily removed, nail flattens.
2-Improvement
3-No change
4-Relapse. Formerly flakey, easy- to-remove fungus
transforms back into a unified, hard, difficult to penetrate
plastic-like wall.
Skin Roughness—Reflects composition's effect on fungus
impregnated skin around the nails.
1-Improvement
1-No change
2-Slight skin roughness was observed
3-Severe skin roughness was observed
Healthy nail appearance
1-Dramatic improvement
2-Improvement
3-No change
4-Relapse. The white edge of the nail widens. Lateral striations
originating at the nail edge begin to intrude on the body of the nail.
Irritation of Skin and Nail
1-Feel nothing
2-Slight pleasant tingle
3-Discomfort
Whenever Stage 4 occurs, or an irritation level 3 was reached,
testing of listed Composition was abandoned.

TABLE 3A

| | Fungus | Skin | Nail | |
|---|---|---|---|---|
| | Quality | Roughness | Appearance | Irritation |
| A | 4 | 4 | 4 | 1 |
| B | 4 | 4 | 4 | 1 |
| C | 1 | 2 | 2 | 1 |
| D | 1 | 1 | 2 | 1 |
| D1 | 1 | 1 | 1 | 1 |
| E | 3 | 2 | 4 | 1 |
| F | 1 | 1-2 | 2 | 1 |
| F1 | 1 | 1 | 1-2 | 1 |
| G | 2-3 | 1 | 2 | 1 |
| H | 2-3 | 1 | 2 | 1 |
| I | 2 | 1-2 | 2 | 1 |
| I1. | 2 | 1-2 | 1-2 | 1 |
| J. | 3-4 | 1 | 4 | 1 |
| K. | 1-2 | 3 | 1-2 | 1-2 |
| L. | 2-3 | 2-3 | 3-4 | 2-3 itch |
| M. | NA | 2 | 2 | 1 |
| N | 1-2 | 2-3 | 3-4 | 1 |
| O | 1 | 2 | 1 | 2-3 |
| P | 1 | 1-2 | 1 | 1 |
| Q | 1 | 1 | 1 | 2 |

TABLE 5
DATA

| | | | |
|---|---|---|---|
| Fungus | | Quantity | |
| under nail | Color of Nail | of Fungus | Relapse |

A. 10% benzalkonium chloride (BC) and benzoyl peroxide (BP)
alone

| | | | |
|---|---|---|---|
| Very easily | Extreme | Increased | Yes |
| Removed | widening of | significantly | |
| | nail crescent | | |

B. 10% benzalkonium chloride and BP w/2% sodium chlorite

| | | | |
|---|---|---|---|
| Very easily | Extreme | Increased | Yes |
| Removed | widening of | significantly | |
| | nail crescent | | |

C. 2% sodium chlorite solution and 2% clotrimazole cream were
mixed in hand then applied to surface, cuticle and the underside of
the nail.

| | | | |
|---|---|---|---|
| Easily | Color | Reduced | No |
| Removed | restores 2 | significantly | |

COMPOSITION D: ONE STEP 2% sodium chlorite and 0.001-2%
Ribose was combined with 2% clotrimazole. Final pH was around 6.

| | | | |
|---|---|---|---|
| Very easily | Color | Reduced | No |
| Removed | restores 2 | significantly | |

D1. COMPOSITION D1: ONE STEP 2% sodium chlorite and 1-2%
Ribose was combined with 2% Clotrimazole. Final pH was around 6.
The resulting cream was combined with 3% hydrogen peroxide

| | | | |
|---|---|---|---|
| Very easily | Color | Reduced | No |
| Removed | restores 1-2 | significantly | |
| White crescent | | | |
| begins to | | | |
| appear | | | |

E. Study 8: Applied Tioconazole 6.5% Ointment (pH 5) AND 2%
sodium chlorite and water solution

| | | | |
|---|---|---|---|
| Keratin | white edge gone, | Thickened | Yes in |
| wall at nail | Nail body | Quick | appearance. |
| Edge | yellow ochre, | recovery | Nail |
| thickens | | | appears |
| | | | bloated. |

F. Miconazole nitrate 2% (pH 3) and 2% sodium chlorite and water
solution. (pH 6)

| | | | |
|---|---|---|---|
| Easily | Color | Reduced | No |
| Removed | restores 2 | significantly | |

F1. Miconazole nitrate 2% (pH 3) and 2% sodium chlorite solution
(pH 6) and 3% Hydrogen Peroxide.

| | | | |
|---|---|---|---|
| Very easily | Color | Reduced | No |
| Removed | restores 1 | significantly | |
| White crescent | | | |
| begins to | | | |
| appear | | | |

G. Nail polish containing Pentavitin and water used in conjunction
with clotrimazole 2%, sodium chlorite solution and glycolic acid
approximately 6%.

| | | | |
|---|---|---|---|
| Removable | Color | Reduced | Uneven |
| | restores 1-3 | | |
| | Uneven | | |
| | recovery | | |
| | Generally | | |
| | improved | | |

H. Nail polish containing Pentavitin and water used in conjunction
with One Step containing 2% clotrimazole and 2% sodium chlorite.

| | | | |
|---|---|---|---|
| Removable | Color | Reduced | Uneven |
| Portions | restores 1-3 | | |
| of nail | (uneven) | | |
| improve | Generally | | |
| | improved | | |

I. Nail polish containing Pentavitin and water used in conjunction
with 2% miconazole nitrate (pH3) and 2% sodium chlorite solution
(pH 6).

| | | | |
|---|---|---|---|
| Very easily | Color | Reduced | Uneven |
| Removed | restores 1-3 | significantly | |
| Came out | (uneven) | | |
| in strips | Generally | | |
| | improved | | |

I1. Method and compositions are the same as in Composition I
except that in place of 2% sodium chlorite solution, a solution
containing 3% hydrogen peroxide at a ratio of 1 part hydrogen
peroxide to 2 parts sodium chlorite solution was substituted.

| | | | |
|---|---|---|---|
| Removable | Color | Reduced | No |
| | restores 2.5-1 | | |
| | Uneven | | |
| | recovery | | |
| | Generally | | |
| | improved | | |

J. Clotrimazole alone

| | | | |
|---|---|---|---|
| No change | discoloration | No significant | Yes |
| | returns | change | |

K. NA to criteria measured by this chart.
L. miconazole nitrate 2%, CS solution 2%, ph 6, under conventional
nail polish

| | | | |
|---|---|---|---|
| No change | Crescent | No significant | Yes |
| | widens | change | |

M. FINGERNAIL 2% Sodium chlorite was dissolved in water and
the pH was adjusted to 6 with glycolic acid. The resulting solution
was combined with 1 part 3% hydrogen peroxide and 2 parts sodium
chlorite solution. NO ANTIFUNGAL ADDED.

| | | | |
|---|---|---|---|
| NA | white edge | NA | No |
| | restored | | |

N. Three PARTS 1% trbinafine gel combined in hand with ONE
PART of a solution containing 2 PARTS 2% sodium chlorite
solution and 1 PART 3% hydrogen peroxide solution.

| | | | |
|---|---|---|---|
| Removable | At first | Reduced at first | Yes |
| | restore 1 | | |
| | After 10+ days, | | |
| | Nail appears | | |
| | to relapse. | | |

O. Ketaconazole 2% and 2% sodium chlorite and water solution
(pH 6)

| | | | |
|---|---|---|---|
| Removable | Color | Reduced | No |
| | restores 2-1 | | |

P. TWO-THREE PARTS 2% Miconazole nitrate to ONE PART
2.10% sodium chlorite

| | | | |
|---|---|---|---|
| Easily | Color | Reduced | No |
| Removed | restores 1-2 | significantly | |

Q. TWO-THREE PARTS 2% miconazole nitrate to ONE PART
2.0% sodium chlorite pH around 8.

| | | | |
|---|---|---|---|
| Easily | Color | Reduced | No |
| Removed | restores1-2 | significantly | |

Terms Defined
Fungus under nail describes the ease with which fungus is excised from under the nail. This treatment generally appears to degrade the self-adhering properties of subungual debris.
Color restores 1: Discoloration fades over entire nail surface, pink begins to appear. White crescent begins to appear
Color restore 2: Discoloration fades over entire nail surface.
Nail Crescent: The distal edged white of the nail. Restoration of clearly delineated nail crescent is an aesthetic objective of this treatment. Widening and diffusion of crescent is typically associated with relapse.
Relapse: means that while treatment progresses, the nail begins to again manifest symptoms (discoloration, thickening, etc) that had already begun to fade with continued use.

### List of Compositions Tested and Observations

A. 10% benzalkonium chloride (BC) and benzoyl peroxide (BP) alone

B. 10% benzalkonium chloride and BP w/2% sodium chlorite

Result: Repeated use for one week resulted in severe relapse and badly roughened periungal tissue; extreme widening of white nail crescent consistent with relapse. Surprisingly, Compositions A and B show promise as effective short-term treatment for keratin removal.

C. 2% Sodium chlorite solution and 2% clotrimazole cream were mixed in hand then applied to surface, cuticle and the underside of the nail.

RESULT: Promising, no relapse; improvement

D. One Step:

COMPOSITION D: ONE STEP 2% sodium chlorite and 1-2% ribose was combined with 2% clotrimazole. Final pH was around 6. It is premixed in a container.

D1. COMPOSITION D1: ONE STEP 2% sodium chlorite and 1-2% ribose was combined with 2% clotrimazole. Final pH was around 6. The resulting cream was combined with 3% hydrogen peroxide at a ratio of 1-3 parts one step to 1 part hydrogen peroxide.

### Summary of Test Results of Composition D See Photos

Subject R: Discernible reduction of expanse of purple nail discoloration and healing of dermataphyte-triggered skin roughness and lesions observable within one week. General improvement in appearance of nail and periungal tissue observed.

Subject C: Excellent

Subject S: Keratin debris was easily removed from the nail after one month of treatment. Discoloration faded. Height of distended nail appears to be reduced.

### Summary of Test Results of Composition D1

Subject R After 17 days of treatment, nail discoloration was dramatically reduced. Crescent begins to refine to a narrower more aesthetically appealing width.

Subject K After three-four weeks of treatment, irregular crescent delineation was firmed to a narrower more aesthetically pleasing result. Discoloration was reduced. Superior result.

E. Study 8: Applied tioconazole 6.5% ointment (pH 5) AND 2% sodium chlorite and water solution (pH 6) then mixed in palm of hand; then blended on toenail.

RESULT: Very good result; quick (within 1.5 hours) establishment of white crescent which was formerly diffuse.

Day 3: (5 treatments) right toe nail appears to swell and bloat. White line of nail crescent gone. Yellow ochre color appears through a large portion of the nail. Keratin wall at nail edge thickens.

CONCLUSION: The result of E (bloating nail, etc) was cosmetically undesirable though it may be effective as an antifungal option.

F. Miconazole nitrate 2% (pH 3) and 2% sodium chlorite and water solution. (pH 6) miconazole nitrate 2% and 2% Sodium chlorite solution (pH 6 were mixed in palm of hand; then applied to cuticle, surface, and underside of affected nails.

RESULT: Subject K: Good.

F1. Miconazole nitrate 2% (pH 3) and 2% sodium chlorite solution (pH 6) and 3% hydrogen peroxide.

2% Sodium chlorite solution was pre-mixed with 3% hydrogen peroxide at a ratio between 1:1-1:4, preferably 1:3 peroxide to SC solution. The solution was dropped onto each affected nail.

Then the SC/Peroxide solution was combined in hand with 2% miconazole nitrate and applied to the surface, cuticle and underside of the nail.

RESULT: Subject K: Excellent on nails. Color restored. Crescent re-established strongly.

G. Nail polish containing Pentavitin and water used in conjunction with clotrimazole 2%, sodium chlorite solution and glycolic acid approximately 6%

Mix in hand and apply 2% SC solution and 2% clotrimazole and 5-7% glycolic acid by to each effected nail after applying 1 drop to each affected nail.

Polish nails/Let dry.

Twice a day, apply SC solution by dropper onto surface and under affected polished nails.

Spread over entire surface of the dried polished nail, under the nail and the cuticle.

Reapply 2% SC solution and 2% clotrimazole and 5-7% glycolic acid over dried polish at least twice a day, as often as 6 times a day.

Continue treatment until polish was removed, then reapply according to above directions, or if nail was left without polish, switch treatment to C, D, D1, F, or F 1.

RESULT: Subject K Inconsistent though generally improved.

H. Nail polish containing Pentavitin and water used in conjunction with One Step containing 2% clotrimazole and 2% sodium chlorite. Method was the same as in example G, but One Step was substituted for Clot and glycolic acid.

RESULT: Subject K Good result. Improvement of portions of nail was noted.

I. Nail polish containing Pentavitin and water used in conjunction with 2% miconazole nitrate (pH 3) and 2% sodium chlorite solution (pH 6).

First apply 2% CS and 2% miconazole nitrate by applying 1 drop 2% sodium chlorite solution to each effected nail. Spread over entire surface of the dried polished nail, under the nail and the cuticle.

Mix one part CS solution to one part 2% miconazole nitrate in hand. Rub into dried polished nail.

Apply nail polish containing water and pentavitin followed by at least twice daily treatment as follows: Apply 1 drop 2% sodium chlorite solution to each affected nail. Spread over entire surface of the dried polished nail, under the nail and the cuticle.

Continue treatment until polish was removed, then reapply according to above directions, or if nail was left without polish, switch treatment to Compositions C, D, D1, F, and F1.

RESULT: Subject K: very good after 4 days without removing polish. Surprisingly, nail improves under polish. Study repeated three times. Positive results are uneven but promising.

I1. Method and compositions are the same as in Composition I except that in place of 2% sodium chlorite solution, a solution containing 3% hydrogen peroxide at a ratio of 1 part hydrogen peroxide to 2 parts sodium chlorite solution was substituted.

RESULT: Subject K: Nail consistently improves when treated according to I1.

CONCLUSION GHI: More consistency and generally improved results may be obtained when a low pH component was included when working with nail polish and CS and imidazoles.

When hydrogen peroxide was added to the sodium chlorite, even at a pH above six; the nail maintains a clearer delineation of crescent and generally appears healthier upon removal of polish.

J. Clotrimazole alone

After initial 11 days of treatment of right foot, Subject R was treated with clotrimazole alone, without CS for 17 days on R1.

RESULT: Subject R Though the periungal tissue improved as expected, relapse of infected nail portion was evidenced by spreading of purple discoloration on toenail. This indicates that clotrimazole cannot penetrate the nail without CS. Treatment abandoned.

K. Nail Polish Stain remover tested. Applied juice of lime and lemon sequentially while gently debriding the nail with a buffing nail tool. After treatment the nail still showed patches of pronounced discoloration. Added 2% CS solution to 1 tsp lemon juice to the nail, then miconazole nitrate 2% to continue onychomycosis treatment.

RESULT: Nail discoloration much improved by morning (approximately 12 hours later).

Treatment was continued with a solution containing 3% hydrogen peroxide solution and 2% CS solution combined at a ratio range of 1:2 to 1:4, peroxide to sodium chlorite. Nail was cleared of discoloration after two weeks twice daily use.

RESULT: Discoloration caused by nail polish staining appears to have been removed.

L. 6-12-10 Miconazole nitrate 2%, CS solution 2%, ph 6, under conventional nail polish (Orly brand) and base coat, (Sally Hansen Brand base and Top Coat) according to Composition I, but with conventional base coat and nail polish in place of nail polish containing Pentavitin.

RESULT SUBJECT K: Toe R 4 of subject K: Good;

All infected toes after 24 hours show improvement. Definition of crescent was clearer on both R1 and L1. Some itching reported on R1.

After one week: R1 and L1 appear slightly relapsed immediately after polish was removed according to Composition K. After 24 hours without polish, and treated twice according to Composition F, (a consistently effective composition) nail appears to manifest symptoms of relapse. L1 develops 3-4 millimeters deep white discoloration below the nail crescent. Nail crescent expands irregularly on R1. More testing needed.

M. 2% Sodium chlorite was dissolved in water and the Ph was adjusted to 6 with glycolic acid. The resulting solution was combined with 1 part 3% hydrogen peroxide and 2 parts sodium chlorite solution. NO ANTIFUNGAL ADDED.

RESULT: Applied to fingernail with irregular crescent delineation and 2 narrow white lateral lines (characteristic of early onset of onychomycosis) intruding approximately 3 millimeters into the pink of the nail. After two weeks of irregular treatment, nail appears improved. Normal appearing crescent edge was re-established. Without continued treatment, no relapse was reported.

N. Three PARTS 1% terbinafine gel combined in hand with ONE PART of a solution containing 2 PARTS 2% sodium chlorite solution and 1 PART 3% hydrogen peroxide solution. Resulting gel was applied to surface, cuticle and under the distal edge of affected nails.

RESULT: Initially good. Discoloration was largely gone. The crescent began to widen after continued treatment and skin appeared dry.

O. Ketaconazole 2% and 2% Sodium chlorite and water solution (pH 6)

1 drop SC was applied to the surface and underside of each affected nail.

Then ketaconazole 2% and 2% sodium chlorite solution were mixed in palm of hand and next applied to cuticle, surface, and underside of affected nails. Good to excellent result.

P. TWO-THREE PARTS 2% Miconazole nitrate to ONE PART 2.10% sodium chlorite solution (ph around 6) was mixed in hand then applied to nail after dropping one part SC under and atop each affected nail.

Good to Excellent result. In slight infection of distal portion of the toenail, discoloration was reduced and crescent was redefined within 8 days.

Q. TWO-THREE PARTS 2% Miconazole nitrate to ONE PART 2.0% sodium chlorite pH around 8 was mixed in hand then applied to nail after applying sufficient SC solution to cover top and underside of the affected nail and surrounding periungal tissue.

Good result

Observations NOTES (informal)

### Study 1:

Effectiveness of benzalkonium chloride (BC) and benzoyl peroxide (BP) alone benzalkonium chloride and BP w/2% sodium chlorite (SC or sc)

### Conclusion:

Benxoyl peroxide and benzalkonium chloride (BC) are effective against nail fungus, softening fungus so that it can be easily removed from beneath infected nails, thus clearing nails of thickened keratin debris and reducing pain from nails distended from fungus infection.

Previous treatment C: 1% CS solution+1% BC suspended after relapse after day 3.

Treatment A: 2% BP and 1% BC.

Treatment w/BP/BC of all 5 toes of left foot

Treatment B: 2% CS+1% BC solution+benzoyl peroxide

Treatment w/BP/BC of all 5 toes of right foot

Treatment 1D: 1% cs solution+1% BC with 2% miconazole nitrate and terbinafine was a slight improvement over Treatment C

Suspended Treatment A after three days.

Skin extremely rough. Nails beginning to get chips; beginning to weaken significantly and breakdown.

Relapse: encroaching whiteness from nail edge back to cuticle. Begins to appear day 2, Becomes unacceptable, covering almost ⅓ nail in irregular pattern by day 3.

Suspended treatment B after 3 days.

### Conclusions/Observations

TREATMENT B: Skin extremely rough. Irregular white crescent.

TREATMENT A: Nails beginning to get chips; beginning to weaken significantly and breakdown.

Relapse: encroaching whiteness from nail edge back to cuticle. Begins to appear day 2,

Becomes unacceptable, covering almost ⅓ nail in irregular pattern by day 3.

Day 4: Treated nails w/treatment G from patent application Ser. No. 12/587,495: 7% Glycolic acid, 2%

CS solution+miconazole nitrate 2%+triple antibiotic in toe sock for one hour.

RESULT: Restoration quite quick.

Treated 11-5 of Subject K with either Treatment C (3 days) or A (3 days) for almost a week. Both treatments involve BC and BP. Severe relapse resulted.

I also used BP and BC on Subject M for speedy softening of keratin debris prior to removal.

Successful result.

### Conclusion for A and B

The combination of BC and BP with or without CS was very effective once a week or once a month.

However when used sequentially for more than one day, twice a day; the nail degrades and softens to consistency of wet cardboard, tears easily. Strength comes back after one month. One month later, nail still cannot be filed—too soft.

IMPORTANT: 2% BP and 1% BC Very effective once a week or month. Quickly softens keratin debris for removal.

RESULT STUDY STOPPED.

### COMPOSITION C

2% Clotrimazole+2% Chlorite Salt, solution NO BP.

### STUDY 3

K Left foot begin treatment of only 0.25-0.5 cm ribbon of 2% clot cream And 1-2 drops 2% sc +1 drop glycolic acid 7% in the form of Proactive Revitalizing toner.

K Right Foot Begin treatment w/2% Bp 2% sc solution 2% Clot

Conclusion: Right and left foot appeared equally effective.

Refined Composition C 2% SC w/0.1% ribose and 2% clotrimazole combined in hand.

### Study 4 Composition D:

FORMULA, One Step

2% clotrimazole, 2% sodium chlorite plus 1-2% ribose, 15 ml clot +0.4 g sc +0.01 Ribose

### Notes:

15 g of 2% clotrimazole was measured

0.4 g of SC then mixed w/15 drops of water.

0.001-0.1 Ribose was added.

Resulting solution was thoroughly stirred into 15 g clotrimazole. The resulting pH was around 6, preferably above six.

Subject K: Results. Product was RUBBED IN to nail. Within 10-15 minutes extremely well-cut delineation of fungus began to soften in on both left and right.

7 hours later nail has continued to improve.

SUBJECT K: Composition was applied for the next six days with good results. At day 4, One Step was rubbed in, and then a thin but discernible layer was applied twice a day for the rest of the treatment period.

Result: More improved.

### Summary of Test Results of One Step—see Photos

Subject R: Discernible reduction of expanse of purple nail discoloration and healing of dermataphyte-triggered skin roughness and lesions observable within one week.

Subject C: Excellent

Subject K: Excellent to fair. There was a wide range of results since a low pH was used which caused dispersal of CS, a key component of the composition. pH needs to be above 6 for stability.

Subject Dr S: Very Good.

COMPOSITION D1 2% sodium chlorite and 0.1% of ribose was added to 2% clotrimazole. Final pH was over 6. The resulting cream was combined with 3% hydrogen peroxide at a ratio of 1-5 parts cream to 1 part hydrogen peroxide and applied to surface, cuticle and under the distal edge of the toes twice a day. Result: Excellent in all tests.

### Composition Study 4

Subject R.

Background: Male Subject R formerly used 3 step process detailed in patent application Ser. No. 12/587,495. After a positive visual result within two months with clear signs that fungus was still present, though was visually much improved, subject R abandoned treatment for approximately 6-7 months. Nails were re-infected/relapsed during that period.

He asked for a simpler treatment regimen, and asked to resume treatment; ONE STEP was developed.

Toes R4-5: Comp C 2% clotrimazole with 2% CS pH 6

Toe R1 Composition J (2% clotrimazole alone) for two weeks, relapse ensued.

Toes R1, 2, 3: ONE STEP

Toes L1: ONE STEP (Composition D) followed by D1

Toes L3-5 Composition C

RESULTS: All treatments except J show improvement. Composition D was preferred. Composition D1 is most preferred.

### COMPOSITION D Subject C

Background: Subject C is a 53 year old female. She estimates that she has had symptoms of Onychomycosis for 2-4 months.

Conclusion: Excellent result.

### Composition O: Subject K

Subject K relapsed after two weeks of treatment w/terbinafine and SC. Ketaconazole and SC were extremely effective. Irritation developed due to an allergy to ketaconazole and treatment was suspended after 10 days.

COMPOSITION P. Good to excellent result. Pronounced “tingling,” not perceived as pain.

COMPOSITION Q. Good result. Pronounced “tingling” was felt that was not perceived as pain; stronger sensation than in Composition P.

### Example 4

### Fungal Inhibitory Concentrations

Trichophyton mentagrophytes and Trichophyton rubrum were grown in broth culture with differing concentrations of sodium chlorite alone, different concentrations of itraconazole alone and combinations of each.

10 mg of 80% sodium chlorite was dissolved in 1 ml DMSO. Additional DMSO was added to dilute the mixture to 1600 micrograms/ml in DMSO, and other lesser dilutions, to form sodium chlorite test reagents. The test reagents were diluted 1:50 with culture broth. Separately dilutions of itraconazole were prepared. The sodium chlorite test reagent/culture broth was diluted 1:2 with the dilutions of itraconazole. Those combinations were diluted 1:2 with broth inoculums of Trichophyton mentagrophytes ATCC MYA 4439, and Trichophyton rubrum ATCC MYA 4438.

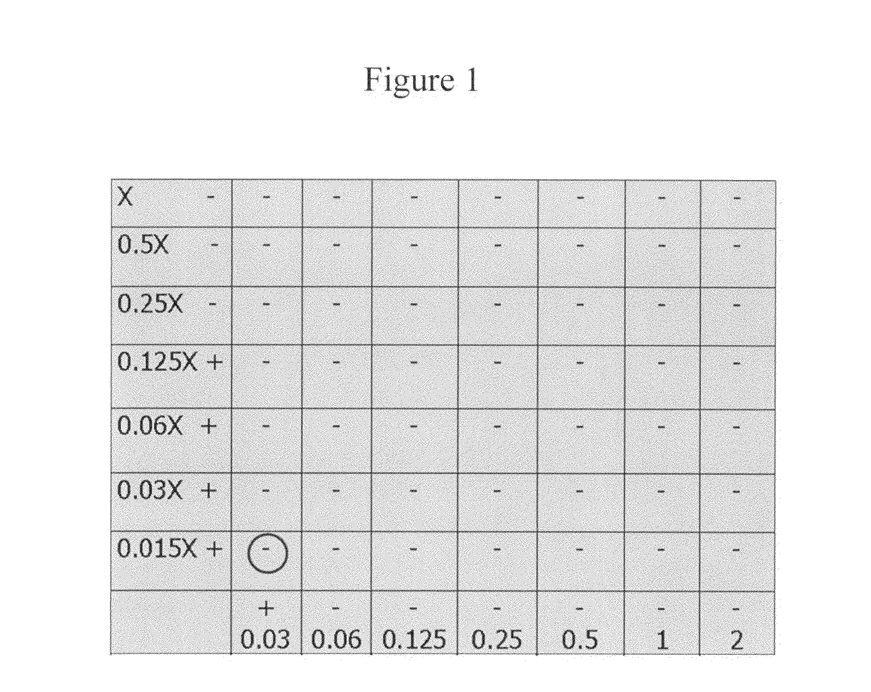

As shown in the examples, in vitro data was generated and the minimum inhibitory concentrations (MIC) tests were determined in broth culture using ATCC MYA 4438. The results are shown in FIG. 1. Itraconazole is the azole used in the study. On the Y-axis are dilutions of the sodium chlorite where undiluted X contains 6.4 micrograms/ml. On the X-axis are dilutions of itraconazole. Fungal Growth is noted by a + and lack of growth by a −.

The MIC of itraconazole alone was 0.06 micrograms/ml and the MIC of sodium chlorite alone was 1.6 micrograms/ml. However, the MIC of itraconazole was 0.03 micrograms/ml or less when combined with 0.096 micrograms/ml or less of sodium chlorite.

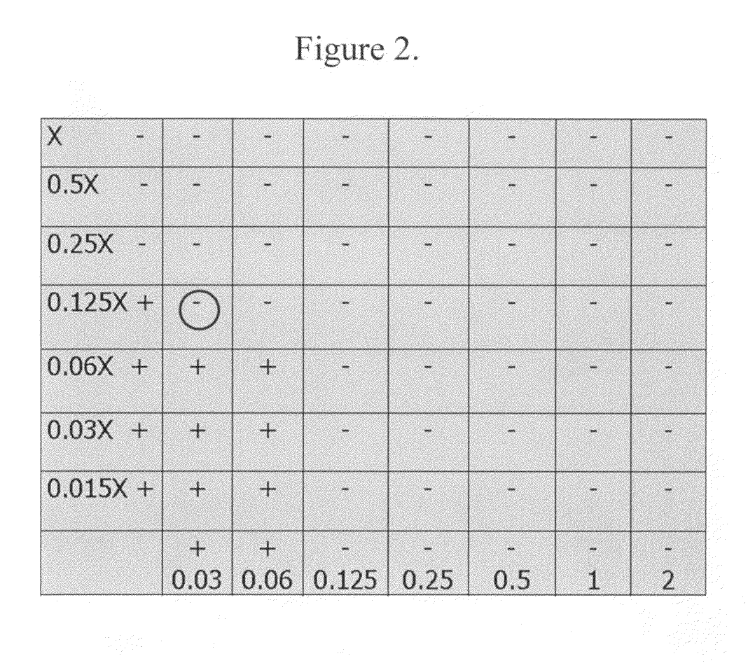

The minimum inhibitory concentrations (MIC) tests were repeated in broth culture using a different strain ATCC MYA 4439. The results are shown in FIG. 2. The MIC of itraconazole alone was 0.125 micrograms/ml and the MIC of sodium chlorite alone was 1.6 micrograms/ml. However, the MIC of itraconazole was 0.03 micrograms/ml or less when combined with 0.8 micrograms/ml or less of sodium chlorite. The results are given in FIG. 1.

For ATCC MYA 4439, the amount of itraconazole required to inhibit the fungus was decreased at least 4-fold from 0.125 ug/ml to <0.03 ug/ml.

For T rubrum, ATCC MYA 4438, itraconazole was decreased at least 2-fold by dropping from a required amount of 0.06 ug/ml to <0.03 ug/ml.

### Example 5

### Compositions Including an Oil

In addition to all of the compositions mentioned above, an oil was added to the treatment for selected sodium chlorite containing compositions. Sunflower, soybean, olive, grape seed and mineral oil were added immediately after 2% sodium chlorite containing solutions with and without 2% clotrimazole (and other antifungal agents). The ratio of oil to sodium chlorite solution was 2:1.

Early clinical data suggests that sodium chlorite used alone with the oil may be almost as effective as sodium chlorite with a conventional antifungal agent for fungus infected toenails.

Example 6

### Skin Treatment

A lesion on a foot was characterized by deep fissures in the skin before treatment. Treatment with 2% sodium chlorite and 2% clotrimazole followed by olive oil in a 1:2 ratio was applied once per day for two days. Some improvement was noted on day 2. One additional treatment was applied and then two treatment were applied every other day with 2% sodium chlorite alone followed by twice as much of a mixture of olive oil and grape seed oil. Further improvement was noted.

### Example 7

### Anti-Candidiasis Treatment

An in vitro determination of the use of sodium chlorite on human tissue culture and broth culture of Candidia albicans was performed. Concentrations of 2% and 0.5% were both effective in killing vegetative Candidia albicans liquid culture. Continued culture without sodium chlorite did not result in regrowth of Candidia albicans suggesting cell killing and (if present) spore inactivation rather than simple growth inhibition.

When tested against human cells in tissue culture, 2% sodium chlorite was shown to be marginally toxic while 0.5% did not display toxicity to the human cells.

TABLE 1
Effects of Sodium Chlorite on Candidia albicans

| Concentration of Sodium | | Candidia albicans |
|---|---|---|
| Chlorite in Culture Broth | Toxicity | killing |
| 0.5% | None observed | Yes |
| 2.0% | Toxic | Yes |

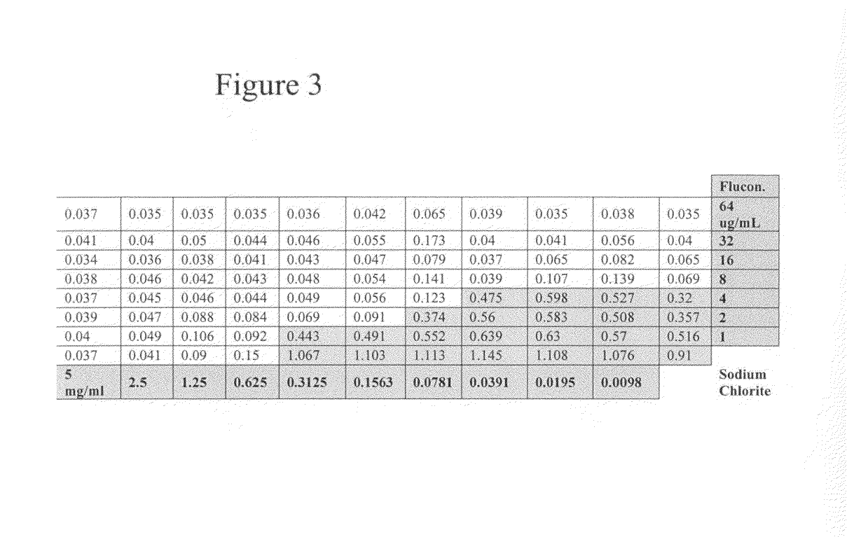

Additional testing was performed using dilutions of sodium chlorite and fluconizole alone and in combination. The data was given in FIG. 3. Growth of Candidia albicans was determined by optical adsorption.

### Example 8

### Photodocumentation

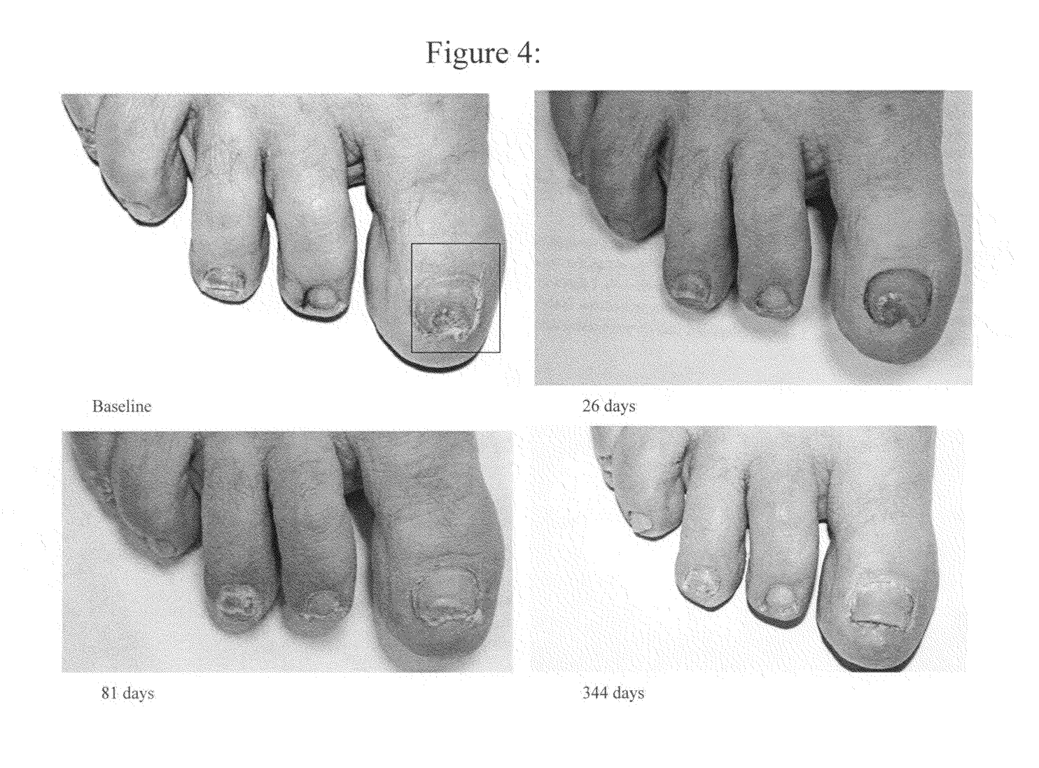

FIG. 4: REGIMEN: Subject S was approximately 65 years old when treated with Composition D twice daily for about 330 days (11 months.) Excellent compliance was noted.

DIAGNOSIS: Positive KOH test before treatment. Negative KOH test result after 11 months of treatment.

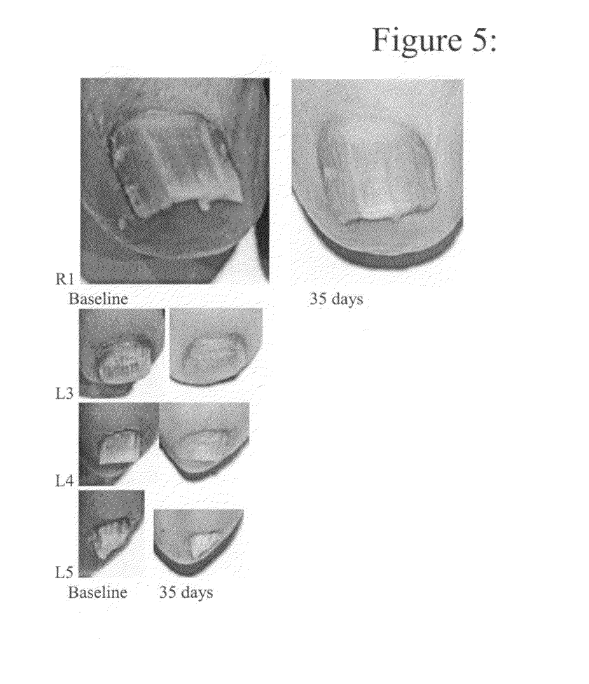

FIG. 5: REGIMEN: Subject J was approximately 67 years old when treated twice daily for 35 days according to P2, Composition D.

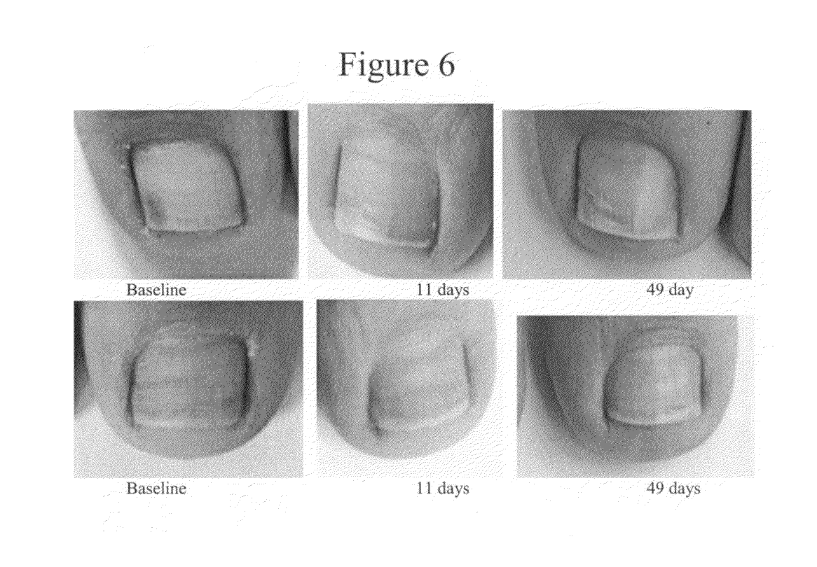

FIG. 6: REGIMEN: Subject C was approximately 54 years old at time of treatment. She applied Composition D twice daily for duration of treatment illustrated.

DIAGNOSIS:. Onset of Onychomycosis symptoms preceded treatment by under 4 months.

It will be understood that various modifications may be made to the embodiments disclosed herein. Therefore, the above description should not be construed as limiting, but merely as exemplifications of preferred embodiments. Those skilled in the art will envision other modifications within the scope and spirit of the claims appended hereto.

All patents and references cited herein are explicitly incorporated by reference in their entirety.

What is claimed is:

1. A method for treating or preventing fungal infections comprising: systemically administering an effective amount of a metal chlorite salt.

2. A composition for treating fungal infections comprising an effective amount of a metal chlorite salt in a sterile carrier for injection or infusion.
